(19) United States
(12) Patent Application Publication
(10) Pub. No.: US 2020/0118643 A1
(43) Pub. Date: Apr. 16, 2020

(54) METHOD AND SYSTEM FOR COMPARING PROTEINS IN THREE DIMENSIONS

(71) Applicant: University of Louisiana at Lafayette, Lafayettle, LA (US)

(72) Inventors: Sumi Singh, Lafayette, LA (US); Vijay V. Raghavan, Lafayette, LA (US); Wu Xu, Lafayette, LA (US)

(21) Appl. No.: 16/654,349

(22) Filed: Oct. 16, 2019

Related U.S. Application Data
(63) Continuation-in-part of application No. 15/725,663, filed on Oct. 5, 2017.
(60) Provisional application No. 62/404,412, filed on Oct. 5, 2016.

(51) Int. Cl. G16B 15/00

(52) U.S. Cl. CPC G16B 15/00

(57) ABSTRACT

A method of comparing three dimensional structure of polymers such as proteins is provided herein comprising the steps of developing at least one key of the protein wherein each said at least one key is based on a quintuple of features consisting of three non-collinear objects in said protein, a representative angle between the three non-collinear objects, and a representative edge length, and comparing the key to either a known database of keys or a key developed for another protein to determine the protein or run a comparison thereof. Additionally, the invention includes a novel method of discovering motifs using key generation. The invention also covers a novel method for representation of protein and drug 3-D structures for the prediction of drug and target interactions.

### CROSS REFERENCE TO RELATED APPLICATIONS

This application claims priority to the Non-Provisional U.S. patent application Ser. No. 15/725,663 entitled “Method and System for Comparing Proteins in Three Dimensions,” filed Oct. 5, 2017, which claims priority to Provisional U.S. patent application No. 62/404,412 entitled “Method and System for Comparing Proteins in Three Dimensions,” filed Oct. 5, 2016.

### STATEMENT REGARDING FEDERALLY SPONSORED RESEARCH OR DEVELOPMENT

Not Applicable.

### REFERENCE TO A “SEQUENCE LISTING,” A TABLE, OR A COMPUTER PROGRAM

Not Applicable.

### DESCRIPTION OF THE DRAWINGS

The drawings constitute a part of this specification and include exemplary embodiments of the Method and System for Comparing Proteins in Three Dimensions, which may be embodied in various forms. It is to be understood that in some instances, various aspects of the invention may be shown exaggerated or enlarged to facilitate an understanding of the invention. Therefore the drawings may not be to scale.

FIG. 1 is rendering of a TSR in 2-D as known in the art.

Table 1 is an embodiment of a method for rule-based label assignment.

Table 2 is a representative Angle Calculation.

FIG. 2 shows an embodiment of a triangular spatial relationship in 2-D for triple (i) in FIG. 1.

FIG. 3 is a depiction of the dataset for angle and length discretization for TSR-3D.

FIG. 4 is a drawing showing DFG motifs as seen in protein kinase.

Table 3 is a chart showing Human kinase dataset (S1).

Table 4 is a chart showing Kinase dataset (S2).

FIG. 5 is a graph showing distribution of selected keys in sequence.

FIG. 6 is a graph showing distribution of angles.

FIG. 7 is a graph showing distribution of lengths.

FIG. 8 is a graph showing sequence cluster sample (S1).

FIG. 9 is a graph showing sequence cluster on dataset (S2).

FIG. 10 is a graph showing structure cluster sample (S1).

FIG. 11 is a graph showing structure cluster sample (S2).

Table 5 is a chart showing paired membership for ideal classification for S1.

Table 6 is a chart showing paired membership for ideal classification for S2.

Table 7 is a chart showing paired membership for S1 as seen from clustering algorithm. Sequence classification is given in lower triangular matrix and structural classification is given in upper triangular matrix.

Table 8 is a chart showing paired membership for S2 as seen from clustering algorithm. Sequence classification is given in lower triangular matrix and structural classification is given in upper triangular matrix.

FIG. 12 is a chart showing DFG motif as seen in sequence alignment of sample S1.

Table 10 is a chart showing structural motifs found in kinase group AGC.

Table 11 is a chart showing structural motifs found in kinase group STE.

Table 12 is a chart showing structural motifs found in kinase group TKL.

Table 13 is a chart showing structural motifs found in kinase group CAMK.

Table 14 is a chart showing structural motifs found in kinase group CMGC.

Table 15 is a chart showing structural motifs found in kinase group TK.

FIG. 13 is a drawing showing TOP 1, TOP 2, TOP 3 search described. Objects with (T) marking are test instances. For black star, the instance with correct class is in TOP 1 of the search. For five-point grey star, the instance with correct class is in TOP 2 of the search. For grey triangle the instance with correct class is in TOP 3 of search.

14) the determination of bin numbers for Theta and Max Dist.

15) an overview of the TSR-based method for protein 3-D structural comparison at global and local levels. a, It shows the steps involved in converting 3-D structures to keys, and objectives of the work; b, All C_{α} atoms were selected from each of the representative 3-D structures, and lengths and angles of all possible triangles (C₃ⁿ) were calculated. Each triangle is converted to an integer (a key) based on its lengths, angles, and amino acids. Consequently, each protein 3-D structure is represented by a vector of integers with their frequencies. A similarity matrix is calculated for clustering proteins, and identical keys with low frequencies in a certain class are found to be the candidates for motifs.

16) the distributions of Theta of 12 protein samples randomly selected from PDB.

17) the distributions of MaxDist of 12 protein samples randomly selected from PDB. Top five MaxDist bin numbers with the smallest variances are indicated.

18) the variances of Theta bin numbers of 12 protein samples randomly selected from PDB. Top five Theta bin numbers with the smallest variances are indicated.

19) the variances of MaxDist bin numbers of 12 protein samples randomly selected from PDB. Top five MaxDist bin numbers with the smallest variances are indicated.

20) a graphical representation of the determination of bin numbers for Theta and MaxDist. a, Top two bin numbers selected from top five bin numbers with the smallest variances for each sample (Samples 1-12) based on the calculations from Theta, MaxDist, all three angles or all three edge lengths; b, The minimum, median and maximum bin numbers of Theta and MaxDist were calculated from the top two bin numbers. The bin numbers with the highest frequency for samples 1-12 are shown; c, The top three bin numbers of MaxDist were chosen mainly based on analyses from a, and b; d, The top four bin numbers of Theta were chosen mainly based on the analyses from a, and b.

21) a representation of one protein (PDB ID: 2HAK) randomly selected from PDB. 35° rotation and/or 5 Å translation were performed. Either rotation or translation yields the identical keys.

22) key generation is independent of rotation and translation, and increases in Theta and MaxDist bin numbers lead to a decrease in number of the keys with high frequency. The graphs show the effect of Theta and MaxDist bin numbers on key frequency was analyzed in four proteins (b, PDB ID: 3KWF; c, PDB ID: 1SB0; d, PDB ID: 2HAK; e, PDB ID: 1EAI).

23) the comparison of protein 3-D structure-based clustering with sequence-based classification. a-b, 16 proteins were randomly selected from four groups: CBP, STAT, Kinase and Protease, and clustered by structural comparison (a) and classified by sequence alignment (b); c-d, 16 proteins were randomly selected from four groups: hemoglobin, cyclin, adenylyl cyclase and CREB, and clustered by structural comparison (c) and classified by sequence alignment (d). a-d, PDB IDs are indicated.

24) the comparison of protein 3-D structure-based clustering with sequence-based classification. a-b, 24 proteins were randomly selected from four groups: glucose transporter, heat shock protein, actin and immunoglobulin, and clustered by structural comparison (a) and classified by sequence alignment (b); c-d, 24 proteins were randomly selected from four groups: RNase, reaction center, transferase and MHC, and clustered by structural comparison (c) and classified by sequence alignment (d). a-d, PDB IDs are indicated.

25) the comparison of protein 3-D structure-based clustering with sequence-based classification. a-b, 24 proteins were randomly selected from four groups: glycerol dehydratase, cyclin-dependent kinase, triose phosphatase isomer, and restriction enzyme, and clustered by structural comparison (a) and classified by sequence alignment (b). a-b, PDB IDs are indicated.

26) the comparison of protein 3-D structure-based clustering with sequence-based classification. a-b, 24 proteins were randomly selected from four groups: retinoblastoma, Ras, epidermal growth factor receptor, and G protein coupled receptor, and clustered by structural comparison (a) and classified by sequence alignment (b). a-b, PDB IDs are indicated.

27) the protein 3-D structure-based clustering of 178 proteins selected from 6 functional classes. 178 proteins were selected from six groups: peptidase, fibroblast growth factor 1 (FGF1), factor X, fructose 1,6-bisphosphatase (F16B), vitamin D3 receptor (D3R) and nuclear receptor coactivator 2 (NRCO2). The method selected the proteins with similar amino acid numbers for each group. Some of the PDB IDs are indicated.

28) a heatmap that shows the cluster of proteases. Dissimilarity values are indicated in the upper left corner in all clustering heatmaps including FIG. 28.

29) a Venn diagram that shows counts of the keys that are specific to each class of proteases, and all possible overlapped regions among protease classes.

30) a dendrogram that shows the clustering of the serine proteases. Number of the proteins in each subclass is indicated.

31) a graph showing the total numbers of the keys and numbers of the specific keys to each class of serine proteases were calculated. Number of the common keys belonging to all classes of serine proteases were calculated.

32) a series of graphs that shows the numbers of the Total, Total Different, Total Common and Total Different Common keys and differences in Theta, MaxDist and frequency between the Total, Common and Uncommon keys of the serine proteases. a, Number of the Total, Total Different, Total Common and Total Different Common keys of the serine proteases; b, Differences in Theta between the Total, Common and Uncommon keys of the serine proteases; c, Differences in MaxDist between the Total, Common and Uncommon keys of the serine proteases; d, Differences in frequency between the Total, Common and Uncommon keys of the serine proteases. a-d, Numbers of the proteins in each subclass of the serine proteases are indicated.

33) the sequence alignment of the representative digestive serine proteases: chymotrypsin, trypsin and elastase, and a representative Triad of chymotrypsin.

34) the sequence alignment of the representative subtilisins, and a representative Triad of subtilisin.

35) the Theta and MaxDist for Triad of serine proteases, and all the triangles formed by Asp, His and Ser of the serine proteases and three sample sets randomly selected from PDB were calculated. Average, standard deviation and the number of proteins are indicated.

36) the representative triangles corresponding to the keys: 7049286, 7174130, 5444573, and 5491202 are shown (PDB ID: 4H4F). Numbers of chymotrypsin-elastase-trypsin out of a total 393 having the keys are shown.

37) the percentage of occurrence of the keys: 7049286, 7174130 5444573, and 5491202 were calculated. Average, standard deviation and number of protein are indicated. Key value difference by 1 (±1) allows minor flexibility for Theta to be considered as presence of the given key. This applies for all other figures where ±1 is specified.

38) a Venn diagram that shows count of the keys that are specific to kinases, Ser/Thr and Tyr phosphatases, and all their possible overlapped regions.

39) a heatmap that shows the clusters of subclasses of kinases and phosphatases.

40) a series of graphs that show the clustering of kinases and phosphatases: A, The total number of the keys and counts of the specific keys to each class of kinases were calculated. Number of the common keys belonging to all classes of serine proteases were calculated and are indicated; B, Numbers of the Total Different, Total, Total Common and Total Different Common keys were calculated. The number of Total Different Common keys was indicated. Average, standard deviation and the number of proteins are indicated. Total keys are defined as all keys including their frequencies. Total Different keys are defined as all keys without considering their frequencies. Common keys are defined as all keys including their frequencies present in every protein of a given protein (sub)class. Different Common keys are defined as all keys without considering their frequencies present in every protein of a given protein (sub)class. These specificities are applied for all other figures. The method designate Common keys as an individual protein level to distinguish from common keys at (sub)class level defined earlier; C, MaxDist of Total, Common and Uncommon keys of kinase classes were calculated. Average, standard deviation and the number of proteins are indicated. Uncommon keys are defined as difference between Total keys and Common keys. The definitions in B and C are applied for all other figures.

41) the differences in Theta (b) and frequency (c) of kinase subclasses. Protein numbers for each subclass.

42) the sequence alignment, and structure, Theta and MaxDist of DFG motif of kinases. a, The sequence alignment of the representative sequences selected from subclasses of kinases. Amino acid sequence similarity and identity are 22.0% and 0.8% respectively. The corresponding PDB IDs for the sequence are indicated.

43) a representative DGF structure (key: 5484102) (PDB ID: 3LB7).

44) a graph representing theta and MaxDist of DFG from the kinases and random sample sets 1-3. Protein numbers are indicated.

45) a demonstration of the method in motif identification and discovery of kinases. a, The representative triangles corresponding to the keys: 8884390 (green), 7192384 (red), 5444573 (pink), and 7173102 (light blue) are shown (PDB ID: 3AXW). Numbers of the kinases out of a total 1,262 having the keys are shown; b, Percent occurrence of the keys: 8884390, 7192384, and 54444573 were calculated. Average, standard deviation and number of the proteins are indicated; c, A representative structure shows two hydrogen bonds: one between Leu301:O and Asp302:N (2.25 Å), and another between Leu301:O and Leu304:N (2.96 Å) that bridges two WDL triangles; d-i, Percent occurrences of U.S. Pat. No. 8,884,390 (green), 7192384 (red), 5444573 (pink), and 7173102 (light blue) in the kinase subclasses: MAK (d), CKII (e), Src (f), cAMPDK (h), CDK (g) and EGFR (i). Number of protein in each subclasses is indicated

46) a series of graphs and heatmap showing clustering, and key numbers and properties of CDKs. a, The heatmap shows structure-based cluster of CDKs; b, Numbers of the total and specific keys for each type of CDKs. Number of the keys belonging to all groups is indicated; c, Numbers of the Total, Total Different, Total Common and Total Different Common keys were calculated; d-e, Differences in Theta (d), MaxDist (e) and frequency (f) between the Total, Common and Uncommon keys of CDK subgroups. Protein numbers for each subgroup are indicated.

47) the specific keys and their structures of CDKs. a-b, Occurrences of three specific keys: 8346432 (AKF) (a) and 5447566/5447567 (DLH) (b) for CDKs, not for other kinase subclasses, are shown. Protein numbers for each kinase subclass and the random samples are indicated; c, A representative structure (PDB ID: 1E1V) of three CDK specific keys and distances between these three keys are shown.

48) the structure-based clustering of the Ser/Thr and Tyr phosphatases.

49) clustering, and key numbers and properties of phosphatases. a, The clustering heatmap shows structure-based cluster of the phosphatases. Different types of phosphatases are labeled; b, Number of the Total, Total Different, Total Common and Total Different Common keys of the Ser/Thr and Tyr phosphatases; c, Differences in Theta, MaxDist and frequency between Total, Common and Uncommon keys of the Ser/Thr and Tyr phosphatases. Protein numbers for each subgroup are indicated in b and c.

50) the phosphatases specific keys and their structures. a, Percent occurrence of the phosphatase specific keys were calculated for presence of at least one key, two keys and three keys. Protein numbers are indicated; b, A representative structure (PDB ID: 1NO6) of three phosphatase specific keys: 2521472 (blue), 4977793 (red) and 8855006 (green) are shown.

51) occurrence and sequence comparison of DFG motif of kinase and phosphatase subclasses. a, Percent occurrence of DFG in the kinases, phosphatases and random samples were calculated. Protein numbers are indicated; b, The sequence alignment of the kinase subclasses. DFG and DWG motifs are labeled.

52) sequence alignment, structure and key properties of WPD motif of the phosphatases. a, The sequence alignment of the phosphatases showing the segment containing WPD, WXDP and DFG motifs; b, The triangles corresponding to WPD (blue) and DFG (red) motifs (PDB ID: 1NO6); c, The structures of DFG and WPD motifs show two hydrogen bonds within DFG motif (2.25 Å between Phe182:N and Asp181:0; 2.25 Å between Phe182:O and Gly181:N) and two hydrogen bonds in WPD motif (2.23 Å between Pro180:N and Trp:O; 2.25 Å between Pro182:O and Gly181:N) (PDB ID: 1N06); d, Theta and MaxDist of WPD motifs of the Ser/Thr and Tyr phosphatases, and random samples.

53) the specific keys of the Ser/Thr and Tyr phosphatases. a, Percent occurrence of the specific keys (7199432, WPD; 8739226, GRG; 8737195, GRH; 4227527, GHQ) of the Tyr phosphatase was calculated; b, Percent occurrence of each key of 8739226, GRG; 8737195, GRH; 4227527, GHQ in the Ser/Thr phosphatases and random samples was calculated; c, Percent occurrence of the Ser/Thr phosphatase specific keys (4230601, HHG; 7072601, HHW; 9102601, HHN) was calculated. a-c, Protein numbers are indicated; d, The representative triangles corresponding to the Tyr phosphatase specific keys: 8739226, 8737195, and 4227527 (PDB ID: 1NO6); e, The representative triangles corresponding to the Ser/Thr phosphatase specific keys: 4230601, 7072601, and 9102601 (PDB ID: 4G9J). d-e, Ratios of number of the proteins that have each of the specific keys over the total number of proteins are indicated.

54) the representative structure of specific keys of Ser/Thr and Tyr phosphatases. a, A representative structure of the Tyr phosphatase specific keys: 8739226 (GRG), 8737195 (GRH), and 4227527 (GHQ) (PDB ID: 1NO6); b, A representative structure of the Ser/Thr phosphatase specific keys: 4230601 (HHG), 7072601 (HEW), and 9102601 (HHN) (PDB ID: 4G9J).

55) the frequency, and structure of two universal keys. a, Percent occurrence of two universal keys: 3803315 (ILL) and 7903915 (VIL) of the kinases, phosphatases, serine proteases and random samples was calculated; b, Frequency of two universal keys: 3803315 (ILL) and 7903915 (VIL) of the kinases, phosphatases, serine proteases and random samples was calculated. a-b, Number of the proteins in each data set is indicated; c, A presentative structure for the keys: 3803315 (ILL) and 7903915 (VIL) of a protein (PDB ID: 1EBB) selected from a random sample is shown; d, The amino acids corresponding to the keys: 3803315 (ILL) and 7903915 (VIL) are from the secondary structures (PDB ID: 1EBB).

56) the properties of two universal keys: 3803315 (ILL) and 7903915 (VIL). a, Theta and MaxDist of the key: 3803315 (ILL) are compared with those of the keys formed from all combinations of Ile, Leu, and Leu; b, Theta and MaxDist of the key: 7903915 (VIL) are compared with those of the keys formed from all combinations of Val, Ile, and Leu; c, Theta and MaxDist from nonpolar and charged triangles of the kinases were calculated.

57) clustering, and key numbers and properties of ArsC and Prdx2 proteins. a, The sequence alignment of ArsC and Prdx2 was performed. Amino acid similarity and identity are 5.8% and 89.8% respectively; b, Clustering of ArsC and Prdx2; c, The Venn diagram shows the numbers of the specific keys and overlapping keys for ArsC and Prdx2; d, Numbers of the Total, Total Different, Total Common and Total Different Common keys were calculated; e, Theta and MaxDist of all, Common and Uncommon keys were calculated.

58) lustering, and key numbers and properties of Hsp70 and Actin proteins. a, The Venn diagram shows the numbers of the specific keys and overlapping keys for Hsp70 and Actin; b, Numbers of the Total, Total different, Total Common and Total Different Common keys were calculated; c, Clustering of Hsp70 and Actin; d, The sequence alignment of Hsp70 and Actin was performed. Amino acid similarity and identity are 5.3% and 89.2% respectively; e, Theta and MaxDist of all, Common and Uncommon keys were calculated.

59) a representation of Arsc, Prdx2, Hsp70 and Actin clusters by Multidimesional Scaling method. Numbers of the distinct and specific keys are indicated.

60) a structure-based evolutionary tree of proteases, kinases and phosphatases. Numbers of the specific keys for each class and type are indicated.

61) a depiction of a small set of specific keys (from three to seven) were identified for CDK2, CDK6, CDK7, CDK8 and CDK9.

62) depicts the method's ability to determine the structure-BLAST search like BLAST search for amino acid sequences, and to study TSR-based protein and drug, and protein and protein interactions

63) depicts an embodiment of the method used for drug key calculations.

64) depicts an embodiment of the method used to identify all amino acids that are likely to interact with drugs.

### BACKGROUND

Proteins are macromolecules or natural polymers with relatively complex structural features. Many of these structural features provide proteins with functional attributes that are vital to biochemical reactions. The primary structure of a protein is its amino acid sequence. A set of 20 amino acids create repeating units within the protein structure. The folding and intermolecular bonding of amino acid units ultimately determine the protein's 3-D shape. Because the amino acid units can repeat several hundred times in a protein, proteins are dynamic and can fold into exceedingly complex shapes. Protein structure studies assist in the investigation of protein-protein interactions and give researchers insight into the biological processes of the cell. By comparing the structure of two proteins, the observer can collect functional annotation, drug-protein interactions, protein-protein interactions and substrate-protein interactions, analysis of active sites, and a plethora of data on critical biochemical activities taking place in a living organism. Thus, protein 3-D structure comparison is an important computational problem that has applications in, e.g., drug design and disease treatment. Developments in this field could lead to cures for a myriad of afflictions, such as cancer, through a better understanding of bio-cell processes.

An important step towards understanding protein functions involves making structure comparisons of a protein under study with proteins stored in the Protein Data Bank (“PDB”), a database of known protein and nucleic acid 3-D structures. As of February 2015, there were nearly 99,133 protein structures freely available in the PDB, which promises to accelerate scientific discovery in all areas of biological science, including biodiversity and evolution in natural ecosystems, agricultural plant genetics, breeding of farm and domestic animals, and human health and disease.

In this way, proteins under study may be arranged so as to identify regions of similarity with data bank proteins that may be of consequence functionally or evolutionarily. This process is called alignment. The degree of structural variation and the inherent flexibility of proteins are critical for their functioning. However, they also lead to enormous amounts of available PDB data. In order to make effective use of this vast amount of data, there is a growing need for more sensitive and automated computational methods for comparing, searching, and analyzing protein structures. Despite active research and the availability of a growing number of methods, there is no widely accepted 3-D structural alignment method. This leaves researchers without a method for searching the PDB with high success rates in finding true matches in the database.

Traditional protein structure comparison or alignment methods can be divided into two main types: sequence-dependent and sequence-independent methods. The results of sequence dependent and sequence independent structure comparison methods are highly correlated, with the exception of the distant homology cases. Sequence-dependent methods of protein structure comparison assume a strict one-to-one correspondence between the amino acids of the two proteins under comparison. In sequence-independent methods, structural superimposition is performed independently, followed by the evaluation of residue correspondence obtained from such a superimposition.

The current sequence-dependent and sequence-independent approaches for protein structure comparison fall into two categories: inter-atomic distance-based and the intra-atomic distance-based. These methods are alignment-based protein structure comparison methods and are based on measuring the distance between two points. For inter-atomic distance-based approaches, the first step is to obtain skeletons for each structure and then select representative points for each skeleton. In the next step, rotation and translation are performed to superimpose points and calculate distance between corresponding points in order to obtain information on protein similarity.

For intra-atomic distance-based approaches, the first two steps are almost identical to inter-atomic approaches: obtain the protein skeleton and representative points. But this family of approaches does not require rotation or translation; instead, it generates a set of matrices representing all the distances between all pairs of points. Structure information is converted to a distance matrix and then a search for similar submatrices is performed. If two matrices are similar, it implies that two structures are similar. If two submatrices are similar, it implies that certain parts of two structures are similar.

These current methods are either computationally expensive because they are based on structural alignment, or do not capture the subtleties of the protein 3-D structure. The inter-atomic and intra-atomic distance methods are both used mainly for global 3-D structure comparison of two or more proteins with similar amino acid sequences and similar size. These methods cannot be used to identify similar local structures if the global 3-D structures of the proteins being compared vary. Also, the methods are incapable of locating sequentially non-conserved, but structurally conserved, subunits of a protein.

A novel method is provided herein that addresses, inter alia, these short-coming by converting 3-D structure information into geometric information, rather than to distance information between two points. The novel method models the global and local structure of proteins in three dimensions. The 3-D modeling is used to compare the structures across proteins using triangular spatial relationship (“TSR”). By doing so, one or more embodiments of the instant method is capable of providing one or more improvements over the prior art, including, in various embodiments: (a) structural representation that incorporates primary structure information from amino acids and 3-D structure information through angular orientation and edge distance; (b) transformation of each structural unit into a unique key via a transformation function that is deterministic, rotation and translation invariant and scale sensitive; (c) design of an approach that leverages the proposed protein 3-D structure representation method to obtain a structural comparison method to discover the conserved structural motifs that are hard to find through sequence alignment; (d) application of the proposed protein structure comparison method in order to find functional clusters and hierarchical classification; and, (e) a fast implementation and querying method to perform protein comparison along with visualization.

The novel method described herein incorporates TSR. TSR has been previously used for 2-D symbolic image comparisons (where each TSR is represented by a quadruple of features). The method modifies the previous 2-D comparison by introducing the concept of scale sensitive TSR 3-D keys that are represented by quintuples of features, and a novel equal frequency discretization method, called Adaptive Unsupervised Iterative-Discretization (“AUI-Dis”), to obtain unique keys. AUI-Dis adaptively chooses the bin (partition representing an interval of values) size to ensure that all the instances of same value occur in the same bin. AUI-Dis iterates over several possibilities of number of bins before it chooses the optimal number of bins that minimizes the variability in bin frequencies. It performs unsupervised equal frequency binning to ensure that the probability of a random variable being located in any one bin is uniform. This feature has previously been unavailable in known equal-width binning algorithms.

After discretizing length and angles of the protein structure, and extracting the quintuples from the protein structure files, the keys and their values are extracted. A key is the result of transforming a structural unit into a unique integer. The key value is the number of times that a unit has repeated in the entire protein structure. The advantage of keys generated using the TSR 3-D algorithm is that it is deterministic and sensitive to scaling, but invariant to rotation and translation. These properties have been proved theoretically and experimentally. These keys are, thus, an accurate representation of protein 3-D structures. The pairwise protein 3-D structure comparison method using keys generated by TSR 3-D can be useful to generate a structural similarity map and to give a ranked similarity output (using, e.g., the Generalized Jaccard Coefficient) by searching a database of proteins with respect to a given query protein structure.

This method is able to accurately quantify similarity of structure or substructure by matching numbers of identical keys between two proteins. The uniqueness of the method includes: (i) structural superimposition is not needed; (ii) use of triangles to represent substructures as it is the simplest primitive to capture shape; (iii) complex structure comparison is achieved by matching integers corresponding to multiple TSRs. The method is used in the studies of proteases, kinases, and phosphatases because they play essential roles in cell signaling, and a majority of these constitute the drug targets.

The new motifs or substructures identified by this novel method (specifically for kinases, phosphatases, and proteases) provide a deeper insight on their structural relations. The method has the potential to be developed into a powerful tool for efficient structure-Blast search and comparison, just as BLAST is for sequence search and alignment.

### DETAILED DESCRIPTION

The subject matter of the present invention is described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies. Although the terms “step” or temporal indicators such as “then”, “next,” etc. might be used herein to connote different components of methods or systems employed, the terms should not be interpreted as implying any particular order among or between various steps herein disclosed unless and except when the order of individual steps is explicitly described.

Furthermore, the described features, structures, or characteristics may be combined in any suitable manner in one or more embodiments. In the following description, numerous specific details are provided, such as examples of algorithms, database sets of proteins, key numbers, key sets, and amino acids. One skilled in the relevant art will recognize, however, that the instant Method for the Three Dimensional Comparison of Proteins may be practiced without one or more of the specific details, or with other methods, components, materials, and so forth. In other instances, well-known structures, materials, or operations are not shown or described in detail to avoid obscuring aspects of the invention. Likewise, although the subject matter is directed to protein comparison, it is understood and intended that this method could be used to analyze and compare a multitude of natural or synthetic polymers or molecules with 3-D shapes other than proteins.

A method for comparing 3-D protein structure is provided herein using a novel, significantly modified triangular spatial relationship (“TSR”) comparison process. The inventive method provides the following improvements: (i) structure superimposition is not needed. It avoids structural rotation and translation, and most importantly, it overcomes the need to compromise between maximizing number of ‘equivalent’ residues and minimizing the RMSD; (ii) inter-chain residue distances (intra-chain residue distances) are based on the distance between two C_{α} atoms (respectively, pairs of atoms within a protein). Since a distance computation does not capture the underlying shape information and the respective amino acids are not included in the distance matrix calculations, motif discovery cannot be easily accomplished by searching for similar distance values. In contrast, triangles are probably the simplest primitives to capture the shape. The method have included amino acids information in the formula of the key calculations to avoid assigning two triangles with similar geometries the same key if any of three amino acids is different between those two triangles. The method allows an effective and accurate identification of similar local structures even when two structures are different at a global level. In addition, the approach can establish whether two or more triangles are connected by a vertex or an edge, thus enabling discovery of more complex shared substructures. In one embodiment, the method enables the identification of common keys present in all proteins of a given class, and specific keys belonging to only a given class, providing a deeper insight into (sub)structural relationships. In this embodiment, a solely structure-based phylogenetic tree can be constructed using identified specific keys for homologous and nonhomologous proteins.

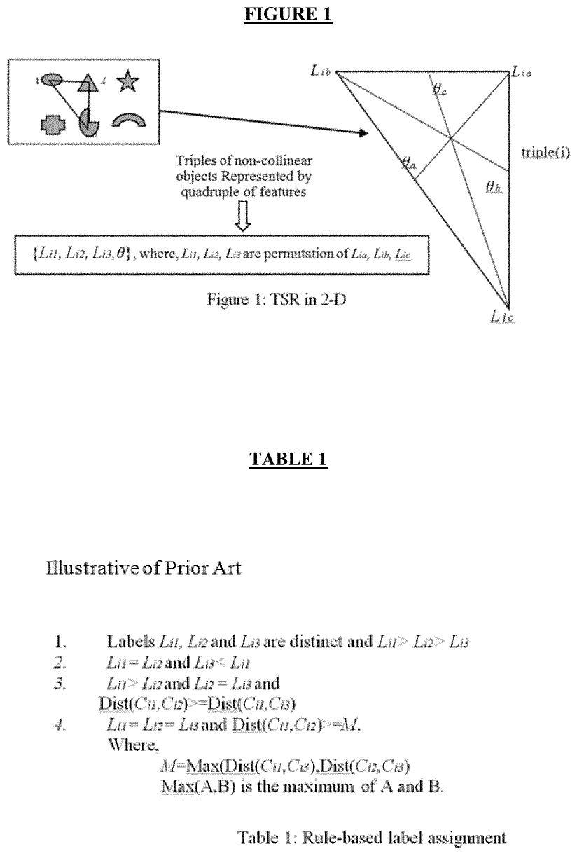

Traditionally, TSR has been used only for 2-D symbolic image representation (i.e., an abstract representation of object relationships in an image). The 2-D TSR method for symbolic images can be used to find global similarity between two symbolic images. The method is based on the relations between three non-collinear objects in a given symbolic image. This local relationship is described by a quadruple of features: three labels used to identify the objects and a representative angle between them (see FIG. 1). TSRs are extracted to generate signatures of images that can be used to establish a signature-based symbolic image database. These image signatures can then be used to compute similarity between two images or retrieve similar images with respect to a query image. The original TSR-based method for similarity computation and retrieval is suitable for symbolic images that have just two dimensions (x, y).

TSRs between objects in a symbolic image are defined by giving relationships between all possible combinations of three non-collinear objects taken in a triple. These objects themselves are represented by unique numerical labels and the spatial relationships between them are represented by an angle (θₐ, θ_{b}, θ_{c}, in FIG. 1). The centroid of these objects form the vertices of a triangle shown in in FIG. 1.

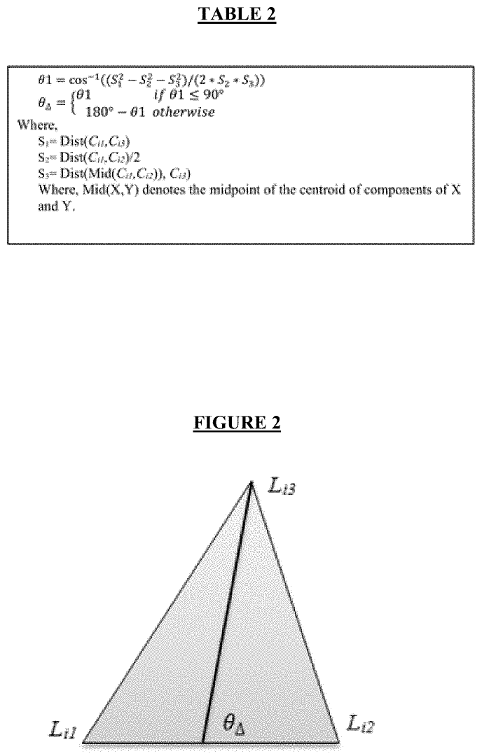

Prior to angle calculations, rule-based label arrangement (according to Table 1) is performed to ensure the uniqueness of the representative TSR. For example, FIG. 2 depicts a triple (i) with the sequence of labels Lᵢₐ=1, L_{ib}=2 and L_{ic}=3. After rule-based label arrangement, the Lᵢₐ, L_{ib} and L_{ic}, are rearranged onto Lᵢ₁=1, Lᵢ₂=2 and Lᵢ₃=3, respectively. Lᵢ₁, Lᵢ₂ and Lᵢ₃ satisfy one of the conditions of Table 1, where i₁, i₂, and i₃ are the corresponding objects in triple and Cᵢ₁, Cᵢ₂, and Cᵢ₃ are the corresponding centroids.

The representative angle is calculated based on label arrangements as given in Table 2. And the TSR between the objects in triple (i) is given by a quadruple of variables {Lᵢ₁, Lᵢ₂, Lᵢ₃, θ_{Δ}} as shown in FIG. 2. This quadruple of variables is used to calculate a unique integer valued key (k) as given by the following transformation function:

k=D(Lᵢ₁−1)m²+D(Lᵢ₂−1)m+D(Lᵢ₃−1)+(θ−1) Equation 1

where, m is the total number of distinct labels; θ is the class value (a “class” as used herein is a set of triples with similar angles or lengths and is also referred to as a “discretization level”) for the class in which θ_{Δ} fails to achieve discretization; and D is the total number of discretization levels. Since all these values are integers, the final key, k, is an integer as well. Integer keys are computationally simpler to work with when compared to the non-integer keys. Each one of the derived keys uniquely identifies a sub-structure.

Representation of 2-D images using TSR results in rotation invariant, translation invariant, and scale insensitive transformation. Although these properties are desirable for image (2-D) representation, they are not all desirable for protein 3-D structure representation. As the sub-structural similarities are indicative of functional relationships, it is problematic if two sub-structures are represented by the same key if they have different sizes or differ in scale. This makes scale sensitivity a desirable property for protein 3-D structure representation. For this reason, significant modifications to the concept of 2-D TSR are required to ensure the representation is scale sensitive.

### TSR-3D

Although TSR has been shown to be useful to represent object relationships in a 2-D image, proteins are 3-D macromolecules, and the structure of a protein is critical to its function and understanding. Therefore, the present invention generalizes TSR 2-D to represent object relationships in 3-D. The novel method described herein uses keys generated through TSR 3-D to compare the 3-D macromolecules. A transformation method that maps the 3-D protein structure information into a vector of keys has been used. These keys together represent an entire protein 3-D structure and can be used to compare 3-D structures of different proteins.

In this one embodiment, a key generation formula for carbon alpha atom, called inter-residue TSR-based keys is used. However, in other embodiments, the details of key generation formula for all atoms of each amino acid, called intra-residue TSR-based keys is used. In other embodiments, this invention provides a method to integrate inter-residue and intra-residue TSR-based keys. In other embodiments still, the invention provides a method for generating intra-molecule keys for pharmaceutical drugs.

For the purposes of illustrating an embodiment of the Method for 3-D Comparison of Proteins, it is of interest to generalize TSR to represent protein structures in 3-D. Proteins are made up of some permutation of 20 amino acids with repetitions. In this embodiment, these 20 amino acids in a sequence are analogized to objects in an image, such that triples of amino acids in the protein 3-D structure of a protein molecule can be considered as the three vertices of a triangle in 3-D. A quadruple of a triple of amino acids of a protein structure in 3-D, obtained using Table 1, Table 2, and Equation 1 as modified herein, can uniquely represent the spatial relationships between those amino acids in 3-D.

Even though proteins are complex structures, the skeleton of the protein is not as complex and still provides the necessary level of sensitivity to identify the protein. This is because each point in the protein structure is represented by the coordinates of the Cα atom (a certain carbon atom joining two amide planes). Each atom is a part of an amino acid. Amino acids bond with each other through peptide linkage resulting in polypeptides or proteins. These linkages or bonds bear specific characteristics of planarity and rigidity and; therefore, have important implication on the structure of a protein by restricting the rotational freedom of the protein to Cα atoms of amino acids. Thus, the skeleton of protein can be adequately used to represent the overall structure of the protein. The protein skeleton is formed by Cα atoms of every amino acid forming the protein. Algorithms that have been established for use in the prior art for finding structural similarity between objects of 2-D images can be transformed to 3-D structures.

TSR for 3-D protein structures is defined by quintuples (rather than the previous quadruple) of features representing triples of amino acids. Before describing the quintuple of features representing a TSR of a protein 3-D structure, it is necessary to define a few concepts and terms. Set x is the set of names of amino acids, where |x|=20. “Labels” are unique continuous numerical values assigned to each amino acid. The set of labels, L, is an ordered set of continuous positive integers and is of same cardinality as x. So that L≤Z⁺, |L|=20, F: x−>L and |x|=20. If, aₖ ∈ x, then, F(aₖ)=Lₖ. A “triple” of amino acids tᵢ ∈ t, belongs to a set of all possible combinations with repetition, of three amino acids so: aᵢₖ, aᵢₗ, aᵢₘ∈x. “Centroid” (C) of an amino acid in triple tᵢ, is given by its representative center, Cα. Often, the centroid is also referred to as the geometric center, center of mass or center of gravity of the object.

The function of a protein changes if the size of the protein is varied. Thus, the novel method takes into account class length. The TSR of the current embodiment includes a quintuple of (five) features. The quintuple includes, three non-collinear amino acids forming the three vertices of a triangle (Lᵢ₁, Lᵢ₂, Lᵢ₃), arranged based on rules given in Table 1. The representative angle, θ_{Δ}, calculated using Table 2 forms the fourth variable of quintuple. A representative distance, D (or “edge length”), for scale sensitivity is given by the distance between Cᵢ₁ and Cᵢ₂. Thus the quintuple of features is given by: {Li₁, Li₂, Li₃, δ_{Δ}, D}. In this way, the key transformation function becomes:

k=θ_{T}d_{T}(lᵢ₁−1)m²+θ_{T}d_{T}(lᵢ₂−1)m+θ_{T}d_{T}(lᵢ₃−1)+d_{T}(d−1)+(θ−1) Equation 2

where m is the total number of distinct labels, θ, is the class value for the class in which θ_{Δ} falls to achieve discretization, θ_{T}, is the total number of distinct discretization level for angle representative, d, is the class value for the class in which D fails to achieve discretization, and d_{T}, is the total number of distinct discretization level for the representative length (or edge length).

Amino acids have natural semantic categorization which can be based on one or more properties such as size, structure, polarity, aromatic, aliphatic, charge, etc. In one or more embodiments, Equation 2 can be modified to reflect a natural categorization of amino acid. Let N contain labels associated with various amino acids categories so that: N⊆Z⁺,ƒ: x N. If, aₖ Πx, then, ƒ(aₖ)=Nₖ. For triple tᵢ, rule-based arrangement of labels of categories is performed as in Table 1 and representative angle calculation is done as described in Table 2. The quintuples of features for generalized TSR 3-D becomes: {Nᵢ₁, Nᵢ₂, Nᵢ₃, θ_{Δ}, D}. Thus, the TSR 3-D key function incorporates the natural semantic categorization of amino acids and is given by the following transformation function:

k=θ_{T}d_{T}(Nᵢ₁−1)v²+θ_{T}d_{T}(Nᵢ₂−1)v+θ_{T}d_{T}(Nᵢ₃−1)+d_{T}(θ−1)+(d−1) Equation 3

where, v is the distinct number categories into which amino acids are grouped. For example, let aliphatic, aromatic, charge, polarity, size, and structure, be the categories into which amino acids can be categorized. The representative positive integer values assigned to these categories could be 1, 2, 3, 4, 5 and 6 in the same order. All the amino acids that are aliphatic will be assigned the label 1, all the amino acids that are aromatic will be assigned the label 2 and so on. The illustrative number of distinct categories or (v) is equal to six. It must be noted, the assumption in this example is that no amino acid may be simultaneous part of two categories.

Quintuples representing TSR 3-D (as given in Algorithm 1) are assigned a unique integer (key) value by using a hash function (a hash function projects a value from a set with many members to a value from a set with a fixed number of fewer members). In one or more embodiments, the functional mapping of TSR 3-D to a key value may be deterministic (two keys will always be the same if and only if the two representative quintuples are the same), insensitive to rotation and translation, and/or sensitive to scaling. Scale sensitivity is introduced so that the TSR 3-D keys represent the structure accurately.

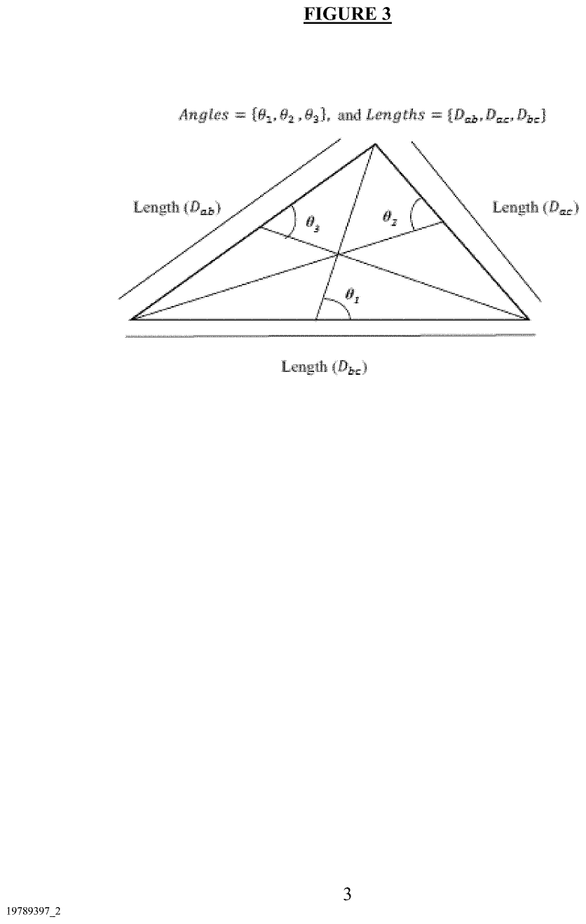

According to Algorithm 1, a set of 20 amino acids is given a single letter abbreviation (A-V). Each amino acid is represented by the x, y, and z coordinate of the centroid as depicted in FIG. 3. A triple of amino acids belongs to the set of all possible combinations with repetition, of three amino acids. Within the triple, a label is assigned to each amino acid. The “labels” are unique continuous numerical values. The set of these labels is an ordered set of continuous positive integers that are have the same cardinality. Next, according to Algorithm 1, rule-based assignment is performed to ensure the uniqueness of the representative TSR. Rule based arrangement is carried out as follows: Let l₁, l₂ and l₃ be the labels assigned to each amino acid aₘ, aₙ, aₚ {where, let m=1, n=2, and p=3} of triple tᵢ. Let d₁₂, d₁₃, d₂₃ be the distance between the respective amino acid centroid. Based on these assignments, one of the equations in Algorithm 1 Step 4 must be met. After performing the rule based arrangement, the representative angle can be calculated according to the formula in Algorithm 1 Step 5. The last step in generating the quintuple of features is to designate a representative length which is the distance between two centroids as shown in FIG. 3. Thus, the quintuple of features generated is: {Li₁, Li₂, Li₃, θ_{Δ}, D}.

### Discretization

After determining the representative length (distance between centroids) and angle (calculated according the equation in Algorithm 1 Step 5) data as described above, that data can be discretized into bins to maximize the coherence of the data grouped together. Those skilled in the art would recognize that there are several methods for discretization when class labels are available, but for data where there is no prior knowledge of class membership, one may use equal width binning or equal frequency binning. The benefit of using equal frequency binning is that there is equal probability of a random unknown instance to fall in any of the bins, reducing extreme biases. A drawback associated with equal frequency binning is the possibility of same observed value to be assigned to different bins because of a sharp cut off as soon as the frequency criteria is fulfilled. Another inherent drawback is the inability of the binning algorithm to place all the occurrences of same value in one bin. To overcome this drawback a new method called adaptive unsupervised iterative discretization (“AUI-Dis”) is used. AUI-Dis ensures that all occurrences of the same value are binned together, while maximizing the bin coherence. In one embodiment, Algorithm 2 is used to find the optimal discretization levels for length and angle using AUD-Dis. Algorithm 2 describes calculating the maximum number of bins to perform iterations using a known formula, computing the expected frequency, minimizing the overall variance of all bins for a given iteration, and choosing the optimal umber of bins for which the partition variance is minimum. D (representative length) and θ_{Δ} (representative angle) are discretized to find the discretization level, d and θ. The result of AUD-Dis is the number of discretization levels and respective bin boundaries, d_{T} and θ_{T}.

In one embodiment, the bin numbers (number of discrete categories) for theta and maxdist are for small size proteins (less than 100 amino acids) in the previous disclosure. In other embodiments, the method provides a method for determining a novel set of bin numbers of theta and maxdist for proteins with amino acids between 200 and 500. Approximately 70% of protein structures in PDB have 200 to 500 amino acids.

The key equation calculated according to the AUD-Dis method and as described in Algorithms 1 and 2 becomes (variables as defined above):

k=θ_{T}d_{T}(lᵢ₁−1)m²+θ_{T}d_{T}(lᵢ₂−1)m+θ_{T}d_{T}(lᵢ₃−1)+d_{T}(d−1)+(θ−1) Equation 2

Once the TSR 3-D keys (k) are computed according to Equation 2, the generated keys are used to compare proteins. The pairwise protein 3-D structure comparison method using keys generated by TSR 3-D can be useful to generate a structural similarity map and to give a ranked similarity output (using, e.g., the Generalized Jaccard Coefficient) by searching a database of proteins with respect to a given query protein structure. The TSR values of two protein 3-D structures p₁ and p₂ are considered as a weighted vector of keys. Equivalence E for a given key kᵢ in two different proteins p₁ and p₂ is defined by Equation 4. The difference z for a given key kᵢ in a pair of proteins is given by Equation 5.

ϵᵢ=kᵢ^{p}^{₁}∩kᵢ^{p}^{₂} Equation 4

zᵢ=kᵢ^{p}^{₁}∪kᵢ^{p}^{₂} Equation 5

The variables in Equations 4 and 5 are: ∩ is the minimum weight of the same keys and ∪ is the maximum weight of the same keys. The Generalized Jaccard coefficient measure is proposed to calculate the similarity between two proteins represented. The Generalized Jaccard similarity coefficient is given by Equation 6, where n is the total number of unique keys in proteins p₁ and p₂, and ϵᵢ and zᵢ are obtained from Equations 4 and 5 respectively.

$${Jac}_{gen} = {\sum }_{i = 1}^{n} {ϵ}_{i} / {\sum }_{i = 1}^{n} {z}_{i} \qquad Equation 6$$

There can be other embodiments, where the individual terms of the summation in the numerator and the denominator are given weights and a weighted summation is done. In one or more embodiments, instead of summing over all n keys, a process for key set reduction may be applied.

### Structural Motifs

In one or more embodiments, the present method may be used to discover and compare structural motifs within proteins. Proteins that are evolutionarily conserved are called homologous. Homologous proteins have been found to have similar overall function. However, at micro level, a set of homologous proteins may exhibit some distinct functionality. The difference in functionality is a result of the presence of a unique functional group that is masked in the overall homology of the proteins. Previous methods of discovering functional groups performed sequence alignment and then looked for conserved groups of amino acids. However, structure is a better indicator of functionality than sequence. Thus, a phylogeny tree (as known in the art) is used for clustering similar protein groups as functional groups.

Experiments were conducted to show that the TSR 3-D keys that follow mean absolute deviation (“MAD”) in a given subset of homologous and distant homologous proteins represent functional groups within that proteins subset. These keys can also be used to find structurally conserved units or motifs. Two sets of protein kinases, the first belonging to humans (Homo sapiens), and the second belonging to various organisms considered distant homologs were tested. The clustering of different functional groups was superior in the homologous proteins compared to that of the distant homologs because the former is more similar in terms of their sequence arrangements. Pairwise correctly and incorrectly placed cluster analysis was performed to compare the sequence and structure clusters. For the two datasets the TSR 3-D-based structure clustering method outperformed the sequence grouping method by 8% and 35%. The TSR 3-D algorithm was tested for its ability to localize the motifs as described below. The algorithm accurately localized the Asp-Phe-Gly (“DFG”) motifs in a group of proteins (DFG proteins belong to the kinase family.). The novel method can also identify local similarity and structural motifs (that is, conserved local sub-structures) within homologous and distant homologous proteins, unlike structure alignment methods.

To test the system, proteins structures are represented using key-value pairs extracted from their structural units. The key is the result of transforming a structural unit into a unique integer as described above and in one embodiment, in Algorithm 1. The key value is the number of times that a unit has repeated in the entire protein structure. Since a protein structure is represented using all possible combinations of triples of amino acids, the number of representative keys per protein structure is relatively high and calls for reduction. Many methods are known in the art to use in conjunction with dimensionality reduction, such as MAD. MAD values are used to identify motifs or portions of a protein shared by all proteins belonging to a class, S. It is based on how much weight values vary for a key within the class. If for a few keys, all proteins of a class have same value of weight, then the deviation in the weight values, as measured by MAD, is zero. Thus, MAD is calculated using the following equations:

$$\begin{matrix}{m}_{k} = 1 / n {\sum }_{i = 1}^{n} {k}^{{p}_{i}} & Equation 7 \\ {MAD}_{k} = 1 / n ( {\sum }_{i = 1}^{n} \vert {k}^{{p}_{i}} - {m}_{k} \vert ) & Equation 8\end{matrix}$$

Where mₖ is the mean for count key k, n is the sample size or the number of proteins in S, k^{p}^{ᵢ} is the weight of key k in protein i of sample S, and MADₖ is the mean absolute deviation of protein k in sample S.

In this embodiment, the keys selected from the reduction are then used for creating clusters of functional groups. These structural clusters can then evaluated against the sequence-based clusters and the former is expected to perform at least to the same degree of accuracy if not higher than the sequence clusters.

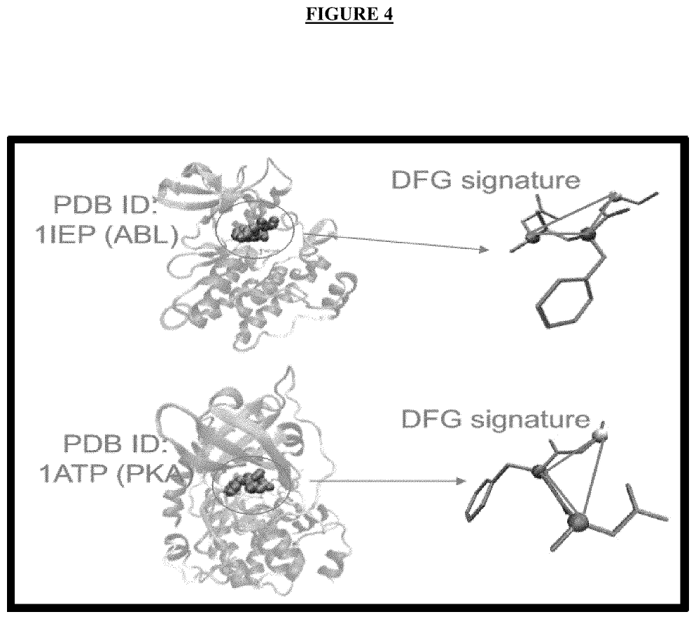

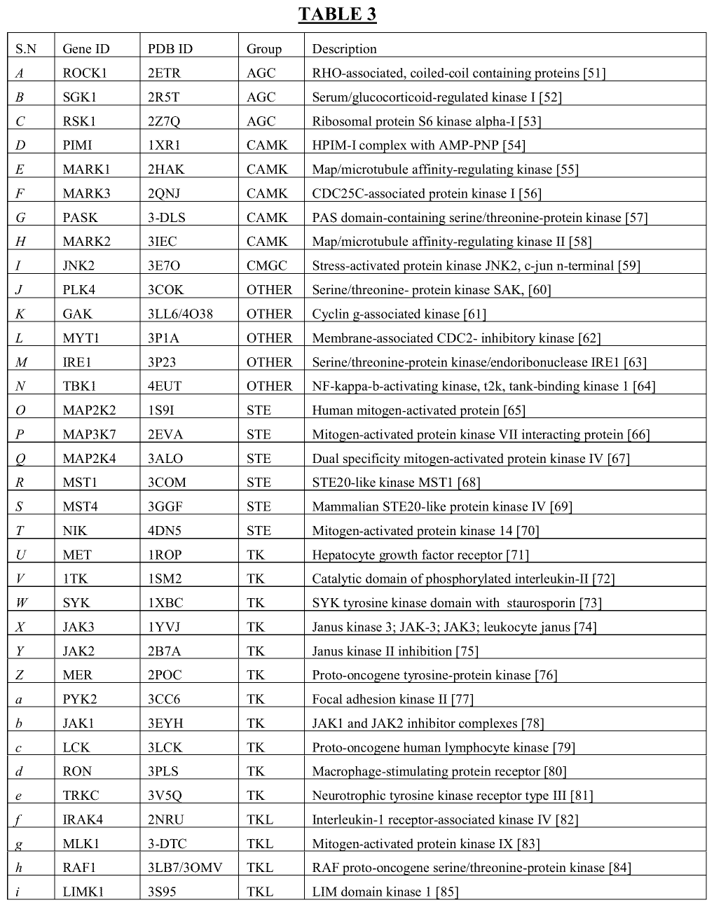

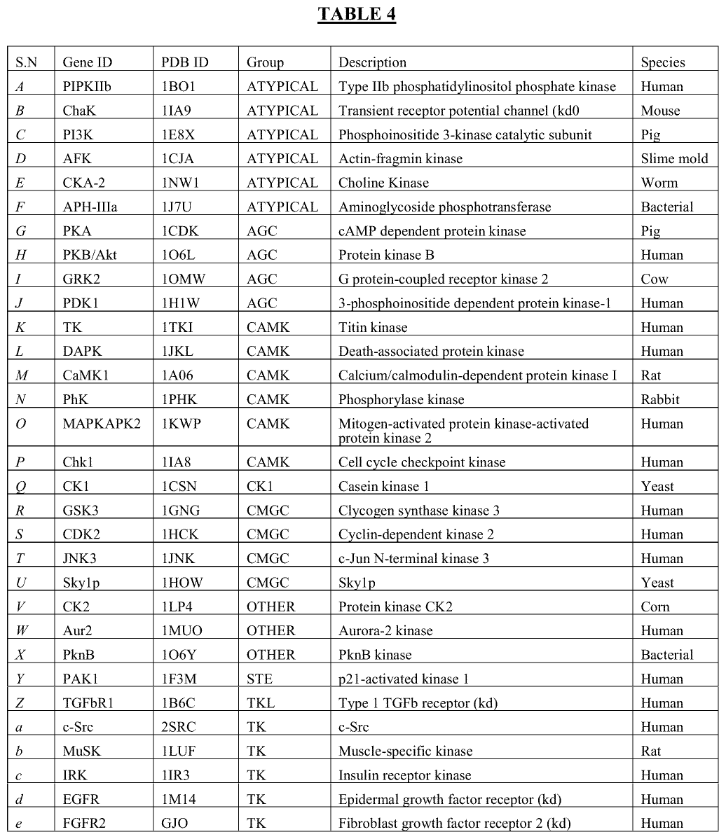

In a majority of protein kinases, there exists a conserved three-amino acid motif at the N-terminal of the flexible activation loop (DFG motif depicted in FIG. 4). This motif is an evolutionarily conserved triple of amino acids. Keys representing evolutionarily conserved functional units, such as the DFG, follow distribution of a low MAD—less than 0.5 across various proteins within the sample. The key representing DFG in a protein kinase also has low frequency of occurrence in each protein in which DFG is present.

MAD is used in the present example because it is a robust estimator of dispersion that is more resilient to outliers in a dataset, although it is understood that other methods and known formulas for estimating dispersion can be used. But with MAD, the effect of outliers is reduced because the deviation from the mean is not squared.

### Example 1

Two-sample datasets from the kinase family have been selected to test the ability of TSR 3-D keys to correctly identify the familial clusters. The first dataset consists of human kinase proteins (“S1”) as set forth in Table 3. S1 is made of randomly selected thirty-five human kinase from PDB. In most protein kinases, a conserved three-amino acid motif, Asp-Phe-Gly (“DFG”) exists at the N-terminal of the flexible activation-loop. S1 was extracted directly from PDB and the chain A was used to establish kinase domain structure. Proteins in the PDB contain one or more polypeptides. Each polypeptide is designated as chain A, B, C, D, E, F, and so on. The 35 human protein kinases (dataset S1) used contain either only chain A or chain A with other chains: B, C, D, and so on. For this specific dataset, chain A is the polypeptide that has kinase activities. S1 was extracted directly from the PDB and the chain A was used for key calculations to represent kinase structures.

The second kinase dataset (“S2”) consists of thirty-one kinases of various organisms. PDB-like structure files for S2 were obtained from the SCOP-ASTRAL 2.03 database. As S2 is taken from a previously published work, no test for percentage sequence similarity was performed on it.

The description of dataset S1 is given in Table 3. The kinase in S2 belong to different organisms is described in Table 4. The descriptions include a unique case-sensitive letter assignment to each kinase in the two samples. Because all the proteins in S1 are human proteins, a description on species is not necessary.

The selection of TSR 3-D keys is important. In this embodiment, the selection is based on the MAD with the parameters that selected keys must pass the maximum requirement of frequency of occurrence in the sample—i.e., document frequency (v) computed based on the number of documents in which the key occurs, and the cutoff, (w) for MAD. The latter is computed using the distribution of the value of the given key across all the proteins in the sample. Algorithm 3 describes one embodiment of the key selection process using MAD. By using MAD, the protein is represented by a lower dimensional vector consisting of locally intersecting keys.

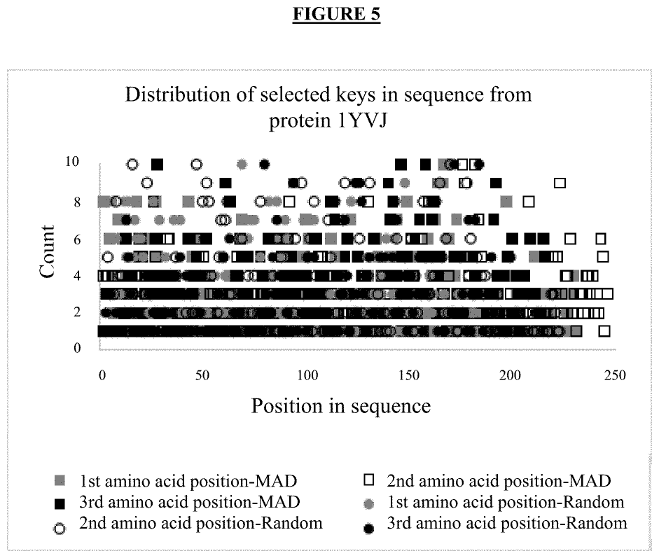

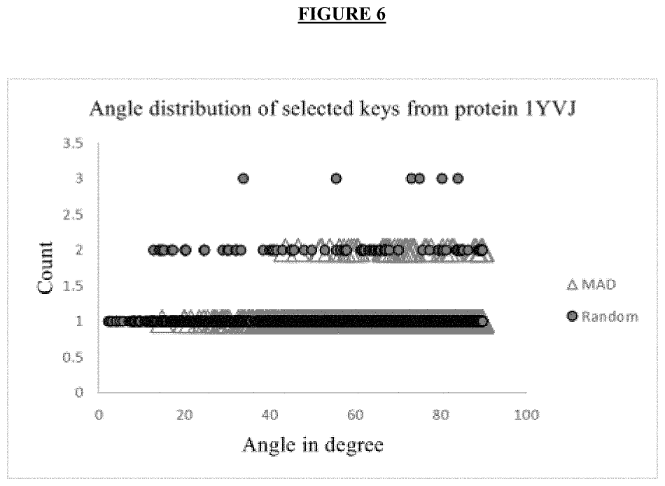

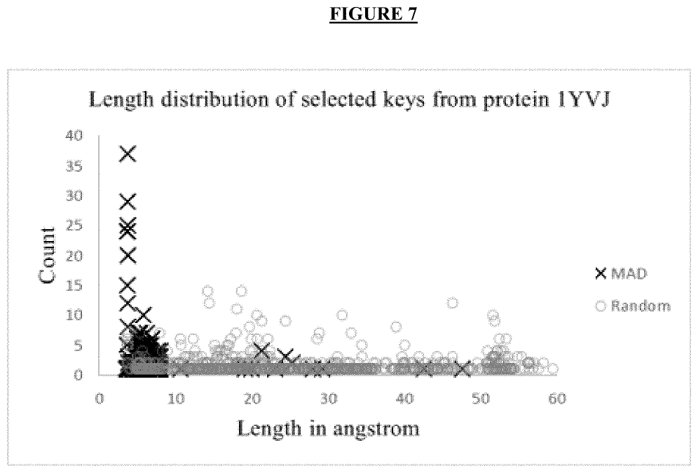

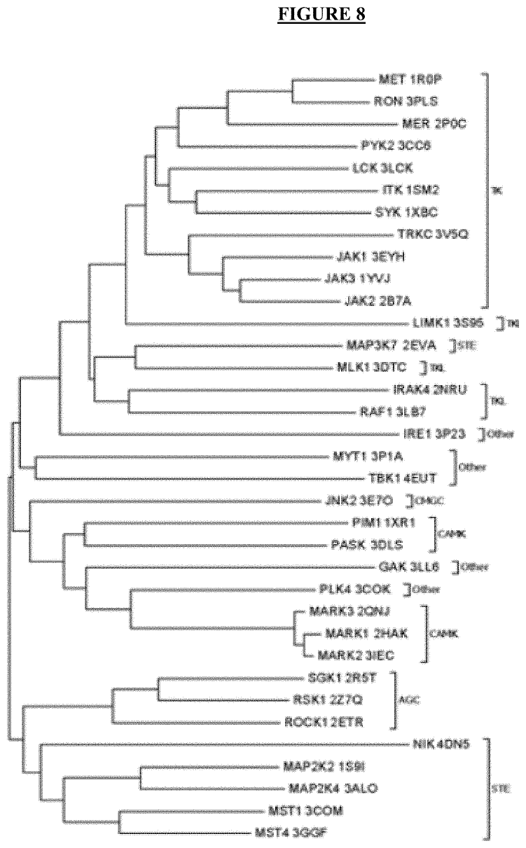

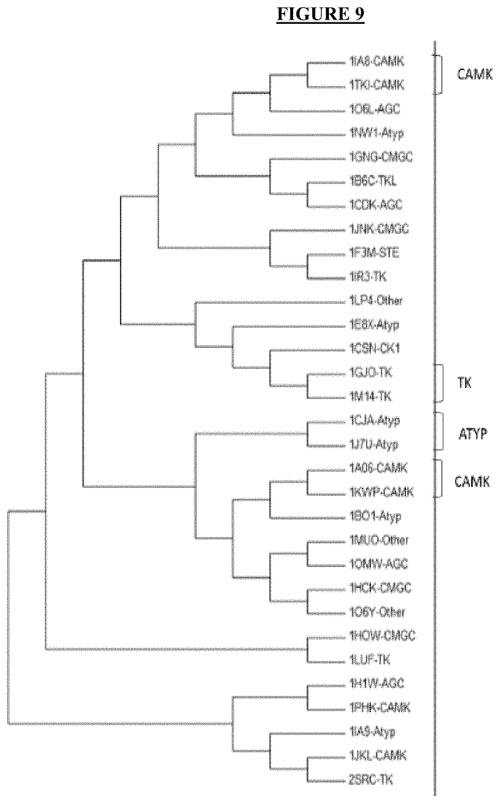

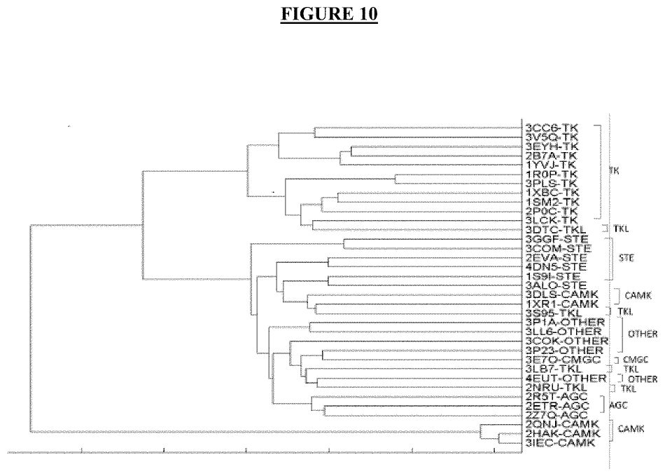

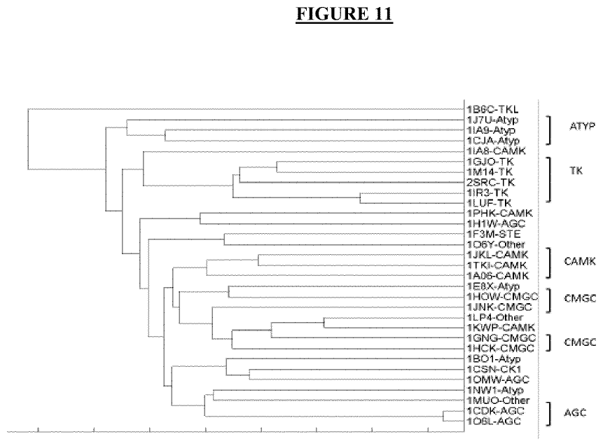

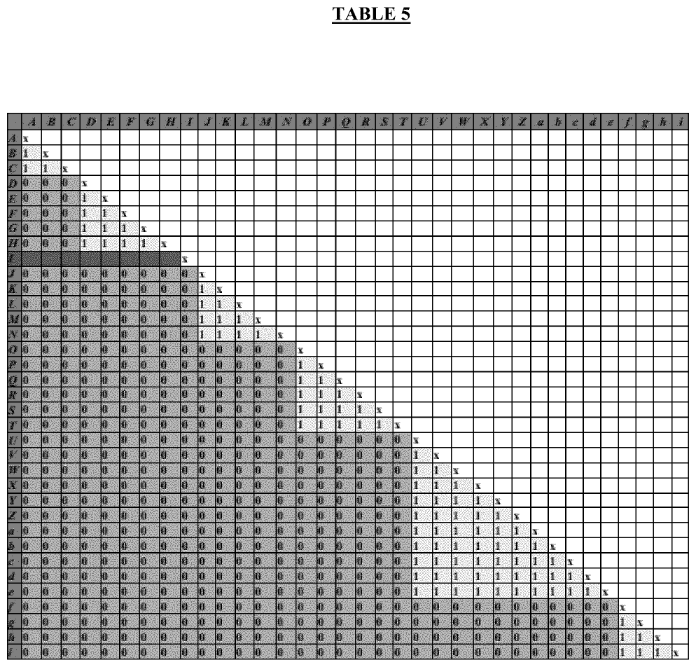

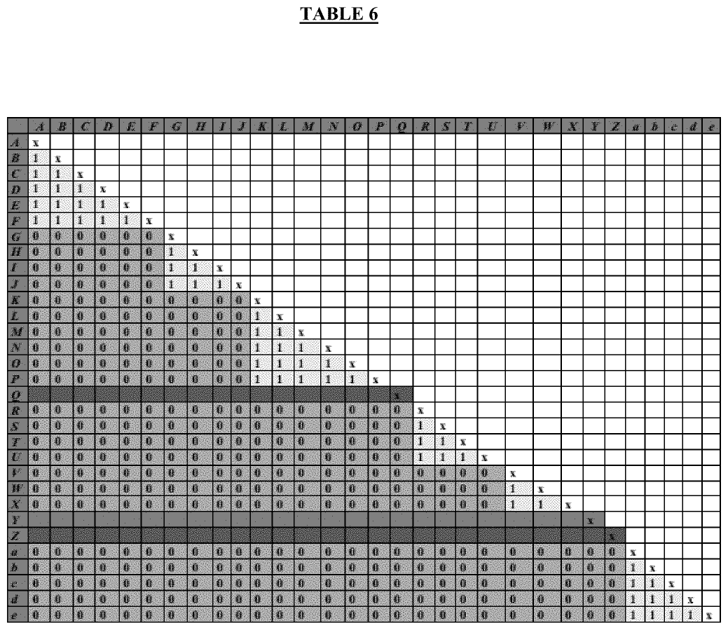

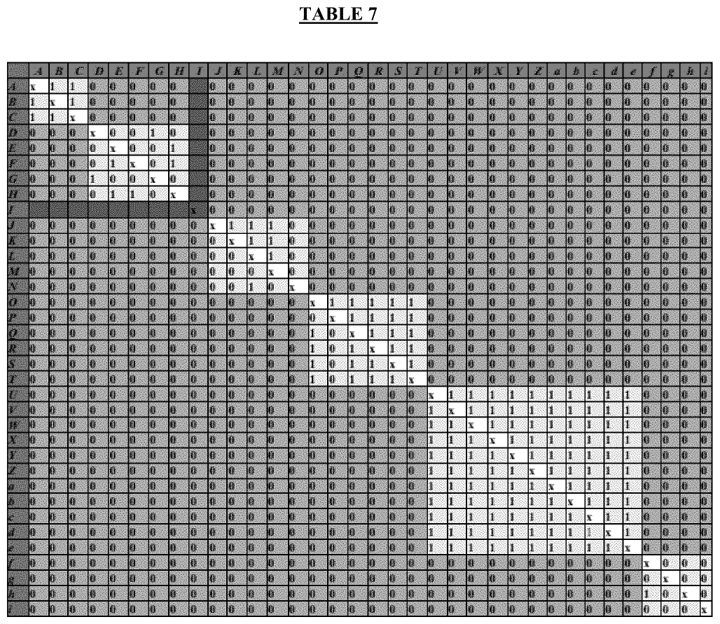

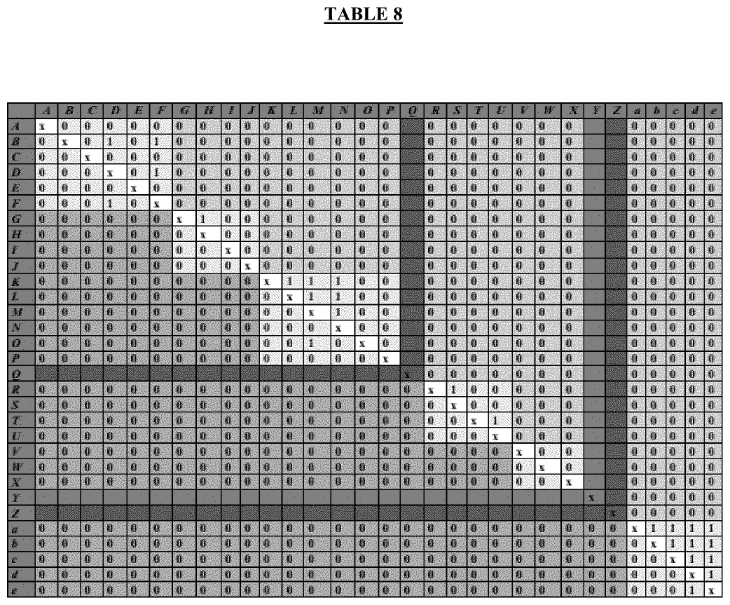

Evaluation of TSR 3-D features that form keys based on MAD criterion against randomly selected keys were performed for keys from four proteins randomly selected from sample S={S1, S2}. FIG. 5 gives the distribution of position of amino acids in the sequence for the selected triples. The distribution of first, second and third amino acids in every selected triple for both MAD is plotted as well as random triples. Similarly, FIG. 6 and FIG. 7 give the distribution of representative angles and lengths of the selected keys. The distributions of amino acids forming the keys in the sequence and the angles representing the corresponding TSR 3-D, were found to be same for both sets of keys as seen in FIG. 5 and FIG. 6.

The distribution of length of the keys selected by MAD were concentrated between 0 to 10 angstrom, whereas for randomly selected keys it was found to be scattered. FIG. 7 shows the distribution of representative lengths for the MAD selected keys (X) and randomly selected keys (o). FIG. 5, FIG. 6, and FIG. 7 show the results from single protein (PDB ID: 1YVJ) for clarity. Similar results were seen with other randomly sampled proteins.

FIG. 8 (for S1) and FIG. 9 (for S2) show the results of the phylogeny trees constructed after sequence alignment. FIG. 10 (for S1) and FIG. 11 (for S2) give the structural clusters formed using MAD keys. The “good clusters” are specified after the square brackets.

The evolutionary grouping of protein kinases are shown in Table 3 and Table 4. The “good clusters” described in FIG. 8, FIG. 9, FIG. 10 and FIG. 11 rely on this prior knowledge and indicate the groups with members that belong together in the ideal cluster.

The paired cluster membership Ø for ideal classification for sample S1 is given in Table 5 and in Table 6 for S2. These cluster memberships are derived from the functional grouping discussed in Table 3 and Table 4. All pairs with membership value of 1 belong to same cluster and those with membership value of 0 belong to different clusters. The rows and columns in Table 5 and Table 6, indicate the protein index, given as serial number (column 1) in Table 3 for S1 and Table 4 for S2.

The ideal classification given in Table 5 for sample S1 is compared to the classification obtained by sequence clusters given in FIG. 8 and structure cluster given in FIG. 10. Similarly, the ideal cluster for sample S2 as given in Table 6 is compared to the sequence cluster for S2 given in FIG. 9 and structure cluster for S2 given in FIG. 11. The results of this comparison are presented in Table 7 and Table 8. The rows and columns of these Tables indicate the protein index for the respective samples.

The comparison of sequence and structure clusters with the ideal clusters for the given samples is performed using the concept of paired membership. For each protein pair as given by the row and column, if in the sequence cluster, its membership is found to be same as the ideal cluster, the pair is given a value of 1, otherwise it is given a 0. A pair is considered to have same cluster membership when they are in same class in both classifications or are in different classes in both classifications. For the simplicity of representation, only those pairs that are expected to be in same class in the ideal cluster are evaluated.

### Cluster Evaluation

Structure is more conserved evolutionarily than sequence is conserved. Structure clusters should, therefore, be closer to the ideal cluster in comparison to sequence cluster. Table 7 and Table 8 give the paired membership for clusters obtained by sequence as well as structure clustering methods for samples S1 and S2. The lower triangular matrices in the Tables mentioned above is of sequence classification and the upper triangular matrix is of structure classification. Evaluation is made with respect to pairs of interest and not all pairs. Interesting pairs are ones that have a paired membership value of 1 in the ideal classification. Similarity (“SIM”) is calculated between sequence classification and ideal, and structure classification and ideal. SIM is given by Equation 9, where Pᵢ, is defined as those pairs of objects that belong to same group in the ideal classification or can be called the ‘interesting pairs’. And Pᵣ is the set of similarly clustered instances from the “interesting pairs” with respect to “good clusters” as given in FIG. 8, FIG. 9, FIG. 10, and FIG. 11. Comparison between the sequence based cluster/tree and

$$SIM = \frac{\vert {P}_{r} \vert}{\vert {P}_{i} \vert} \vert SIM ∈ [ 0 , 1 ] \qquad Equation 9$$

structure based cluster/tree is made with respect to the “goodness” of clustering according to Equation 10:

k=Pᵢ(y)

k*=Pᵣ(y*)

k**=Pᵣ(y**)

c*=SIM(k*,k)

c**=SIM(k**,k) Equation 10

where, y is the ideal classification, y* and y** are two classifications under examination. Here y* will be considered a better classification/tree if c*>c**, as y* is closer to ideal, or vice versa. For the calculation purpose, all the objects in a sample that resulted in singleton cluster in the ideal cluster were not included—i.e., all the objects of classes which have no more than one object were ignored.

Table 9 below compares the structural classifications with ideal, and sequence classification with ideal. Sample S1 has similarity value of 0.75 to ideal for sequence clustering, and 0.83 to ideal for structure clustering. For homologous human kinase proteins in sample S1, the structure clustering using MAD selected TSR 3-D keys outperforms sequence clustering, but the difference between the similarity values is relatively low.

TABLE 9
Comparison of clustering methods

| | S1 | | S2 | |
|---|---|---|---|---|
| \|Pi \| | 99 | | 55 | |
| | Sequence | Structure | Sequence | Structure |
| \|Pr \| | 74 | 82 | 3 | 22 |
| Similarity with ideal (SIM) | 0.75 | 0.83 | 0.054 | 0.40 |

For sample S2, the similarity for sequence clustering to ideal is 0.054 or 5.4%. The similarity of structure clustering to ideal is 0.40 or 40%. Although, these similarity values are much less compared to those seen for S1, structural clustering using MAD selected TSR 3-D keys completely outperforms the sequence.

### Use in Structural Motif Discovery

Keys that fulfill the MAD criteria can be used to find structural motifs. Some of these evolutionarily conserved sub-structures may be found in sequence alignment. Structural motifs can be defined by its smallest TSR 3-D unit that is by a triple of amino acid, or by longest sub-structure. In both the cases the amino acids being represented by the sub-structure of interest, may or may not be continuous in the sequence.

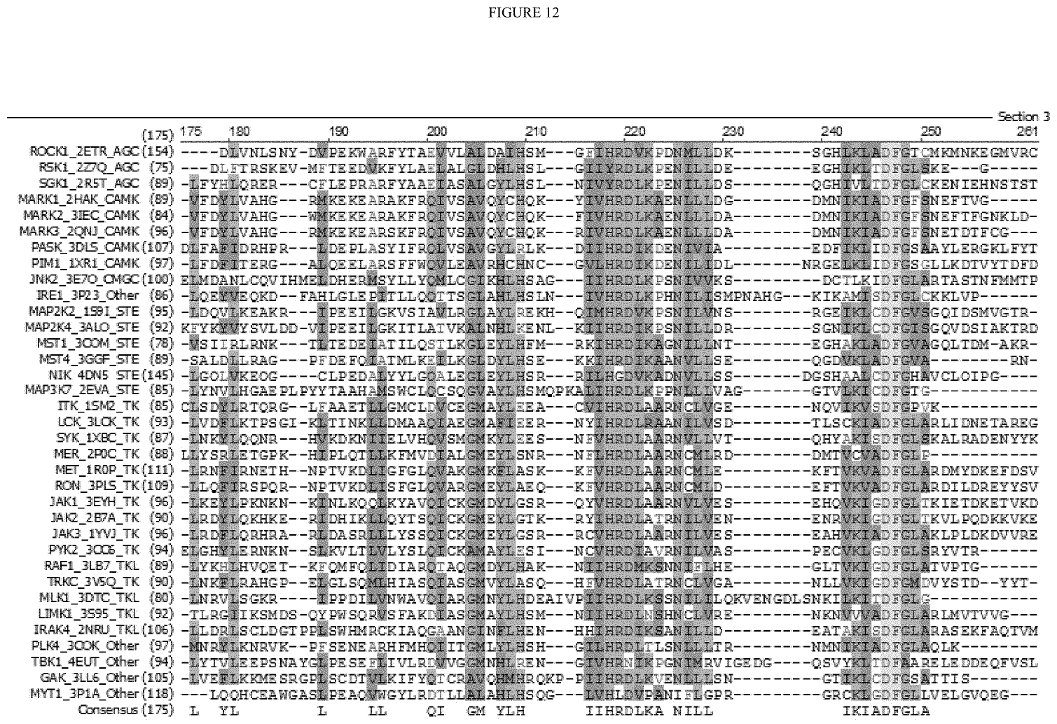

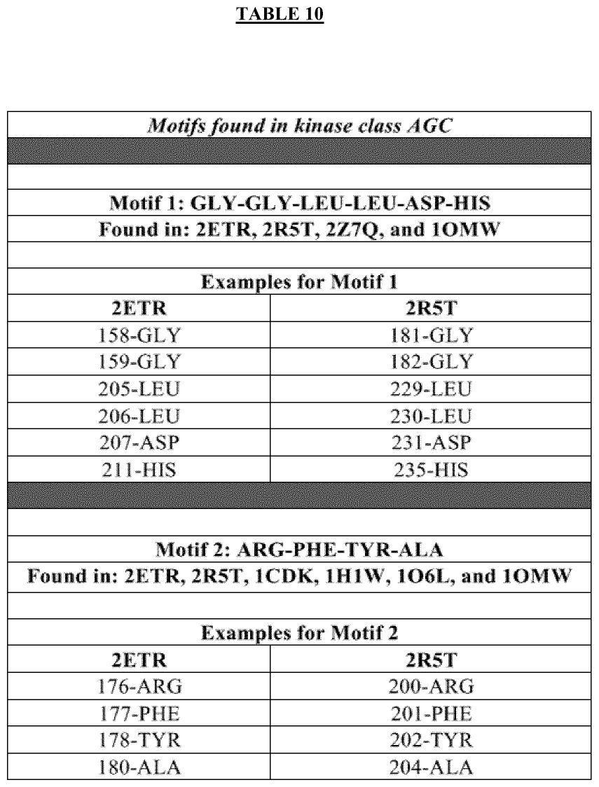

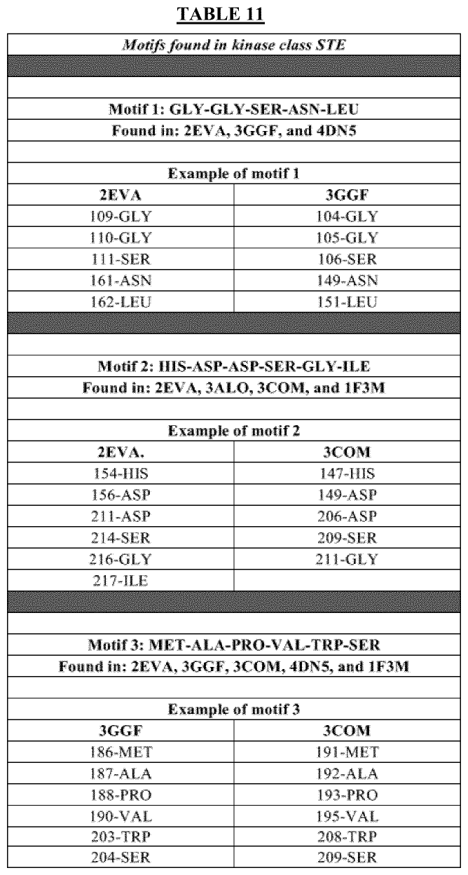

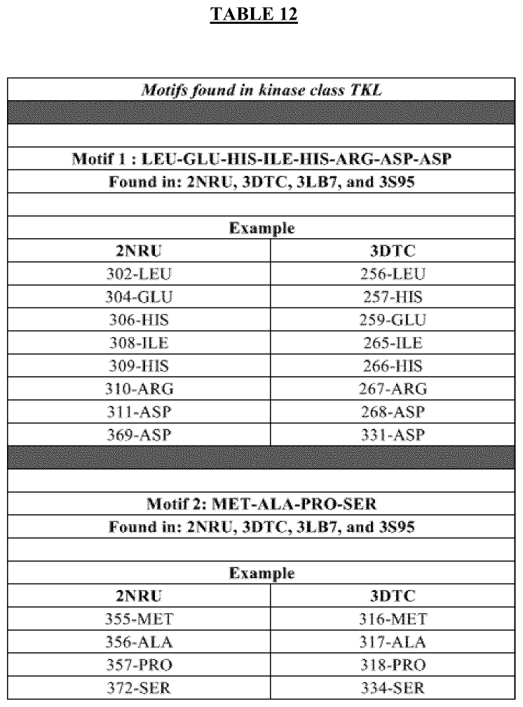

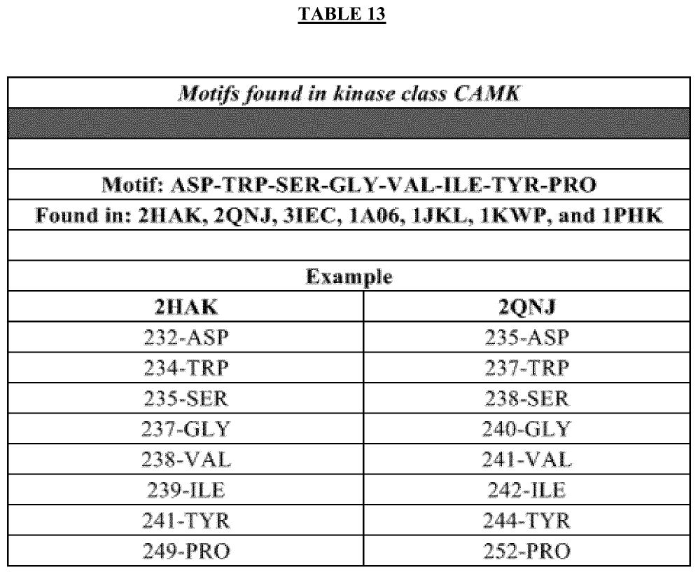

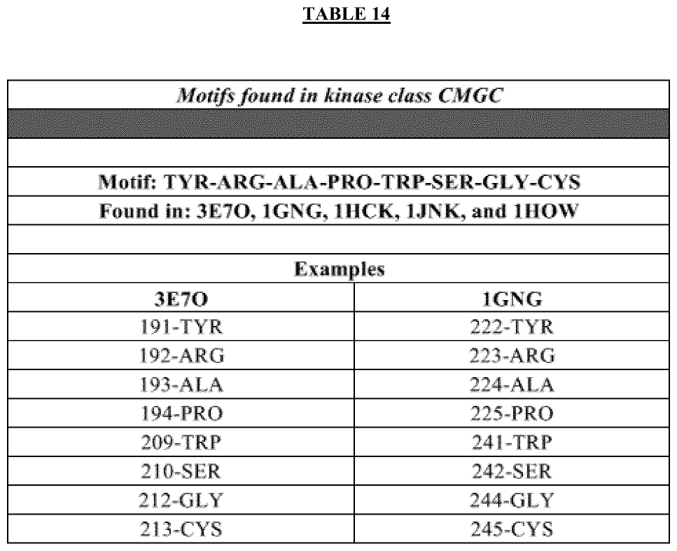

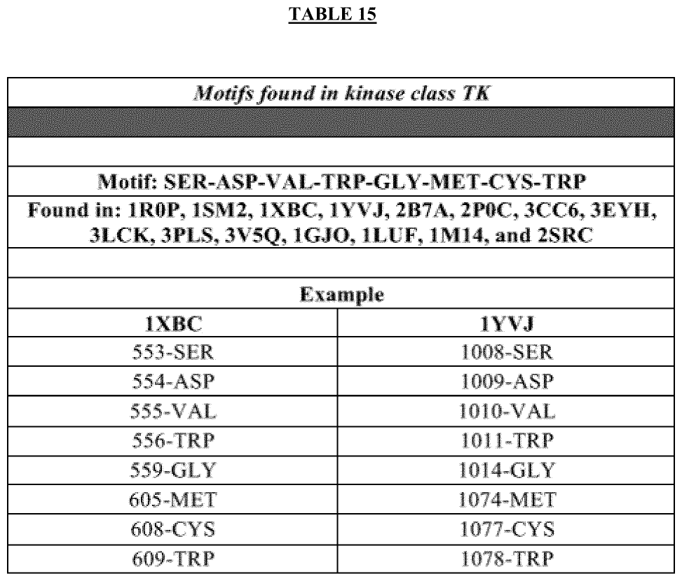

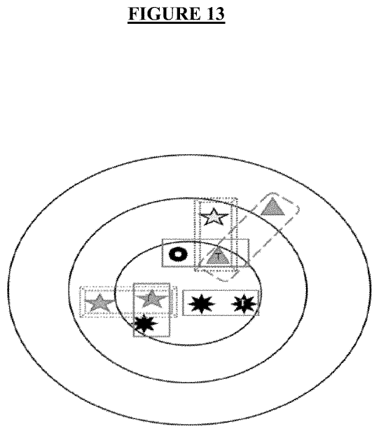

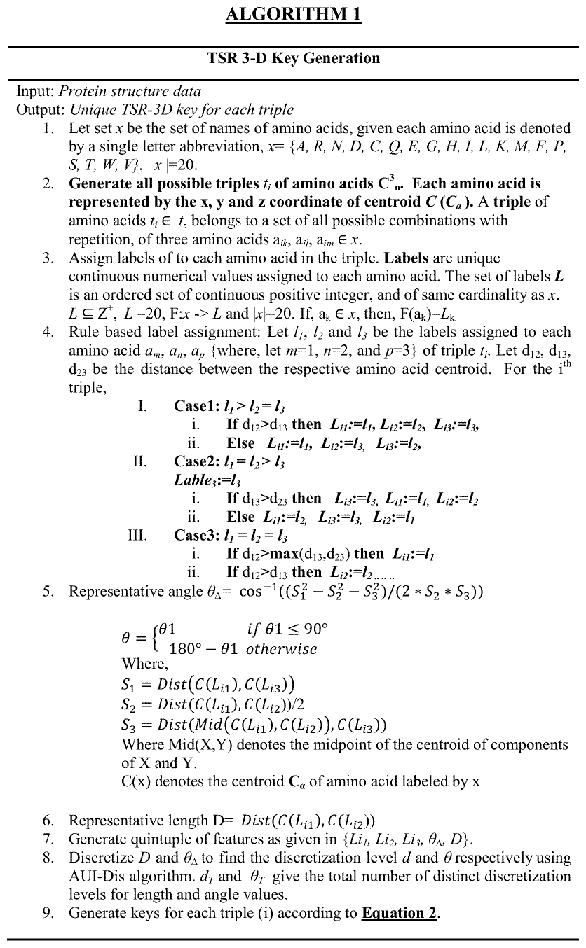

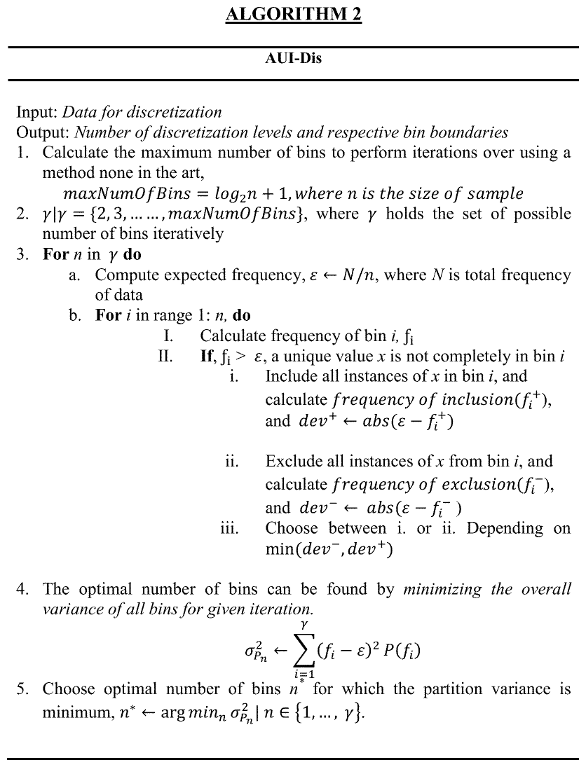

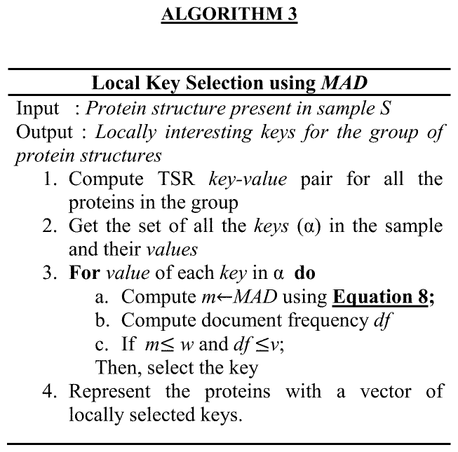

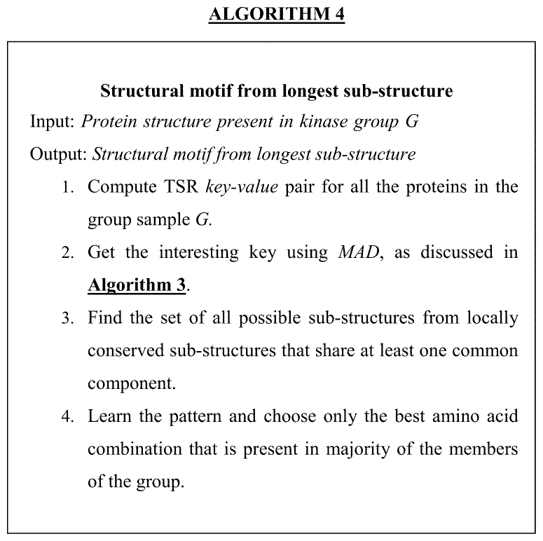

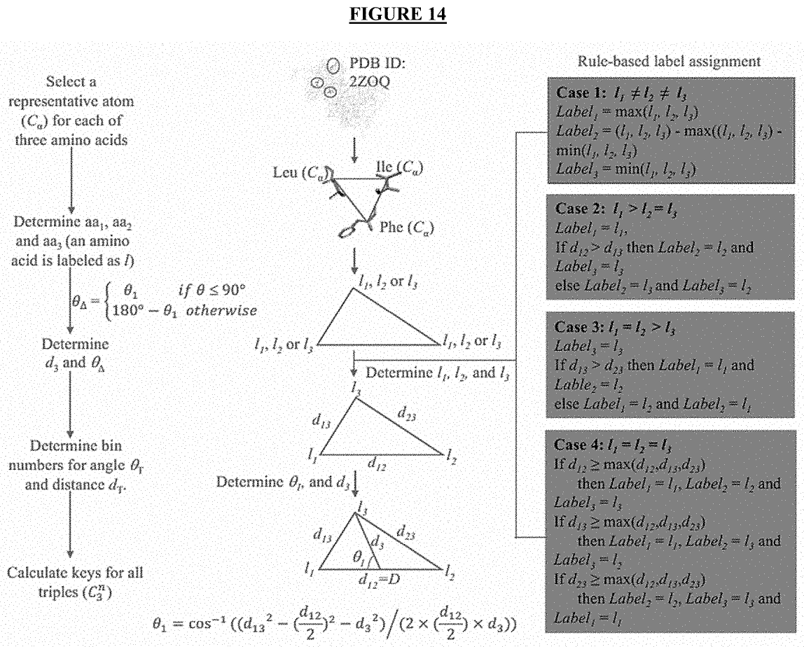

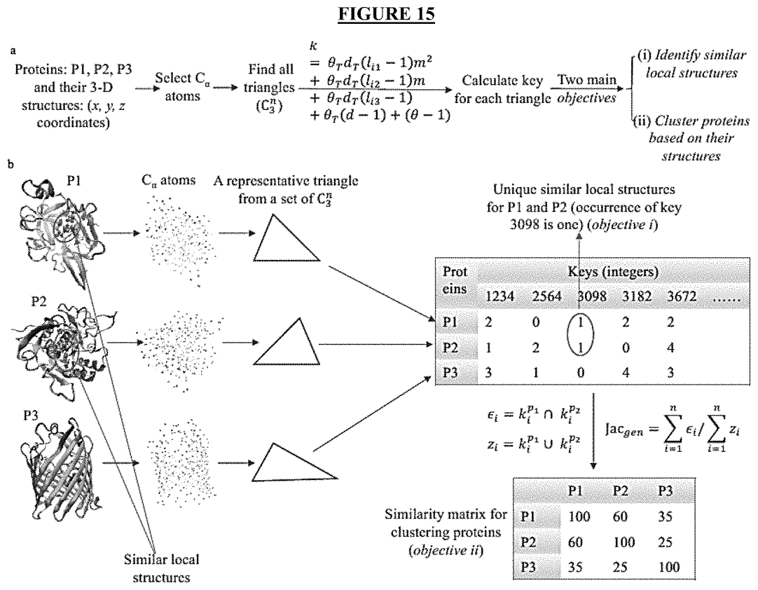

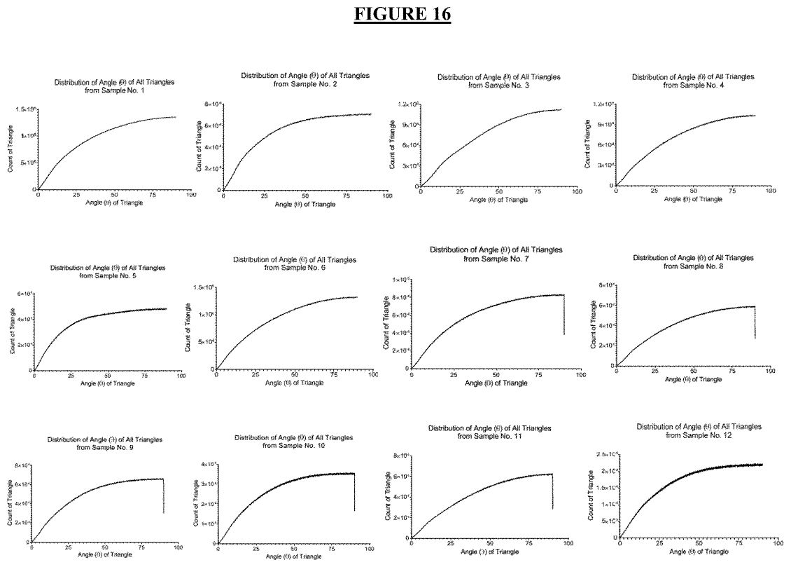

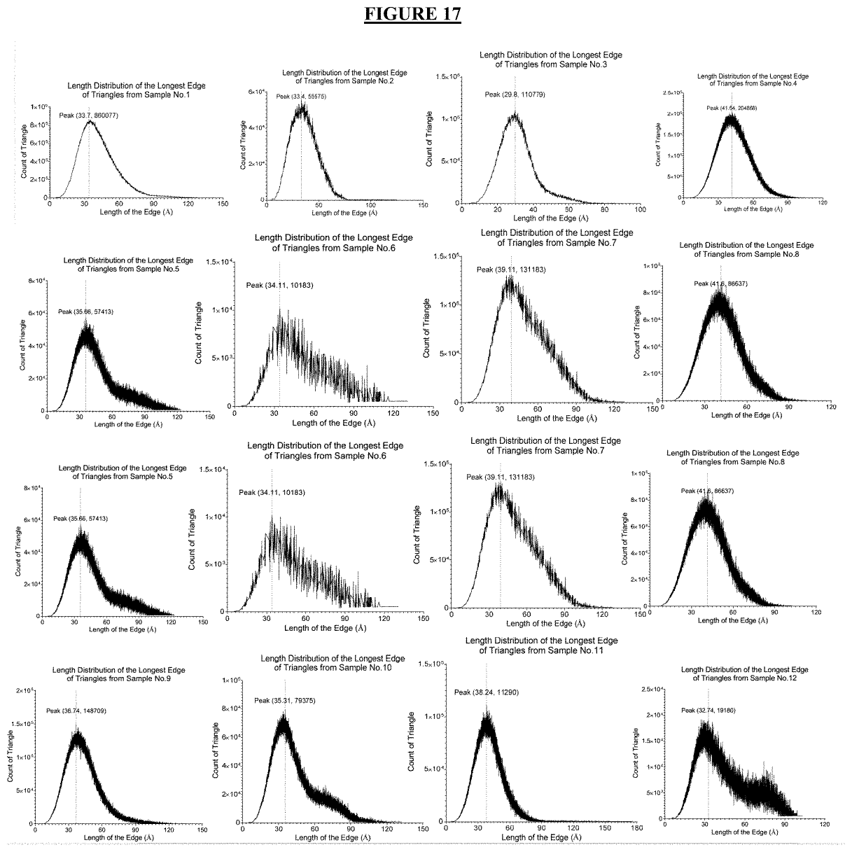

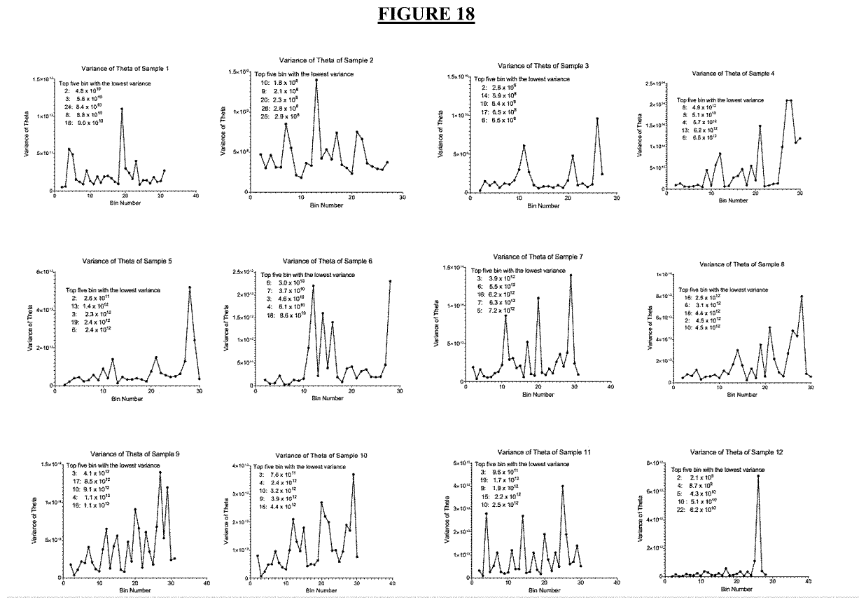

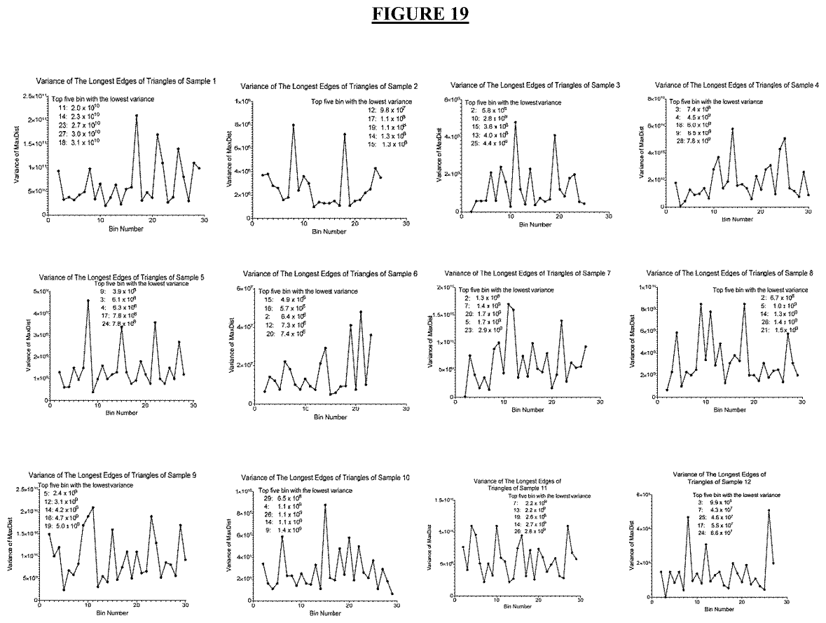

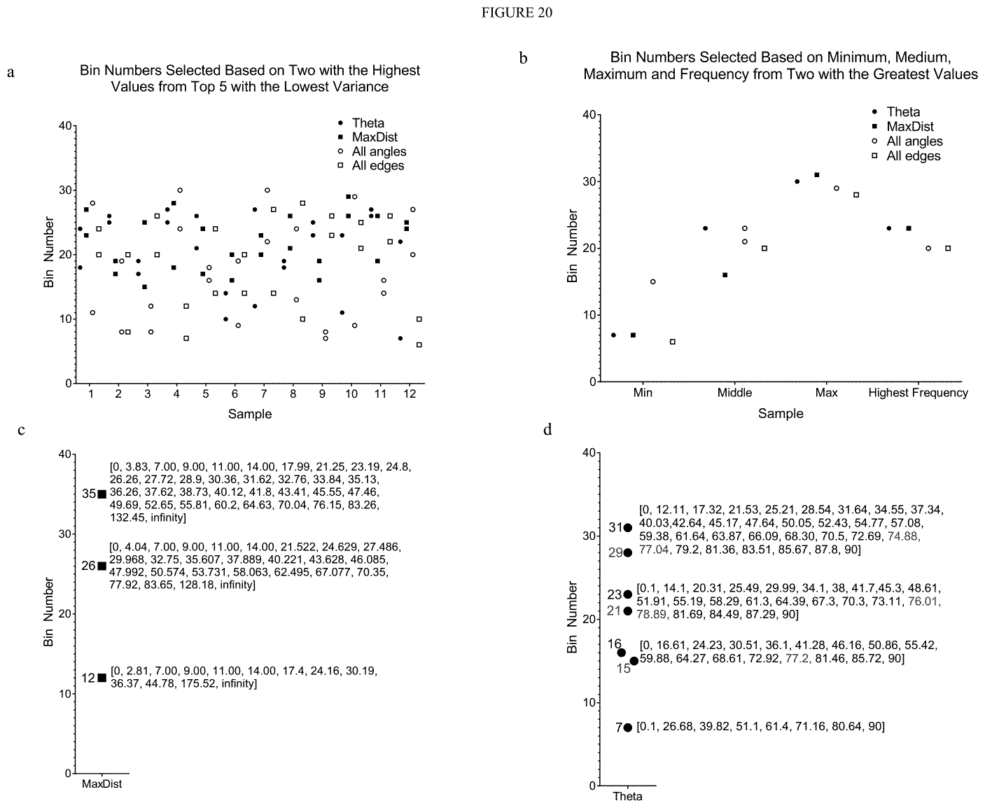

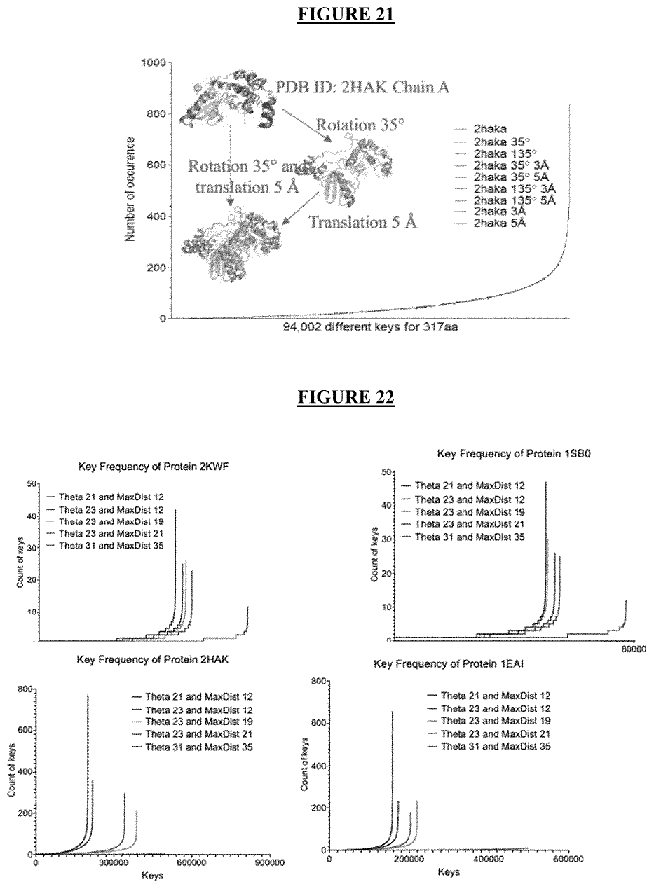

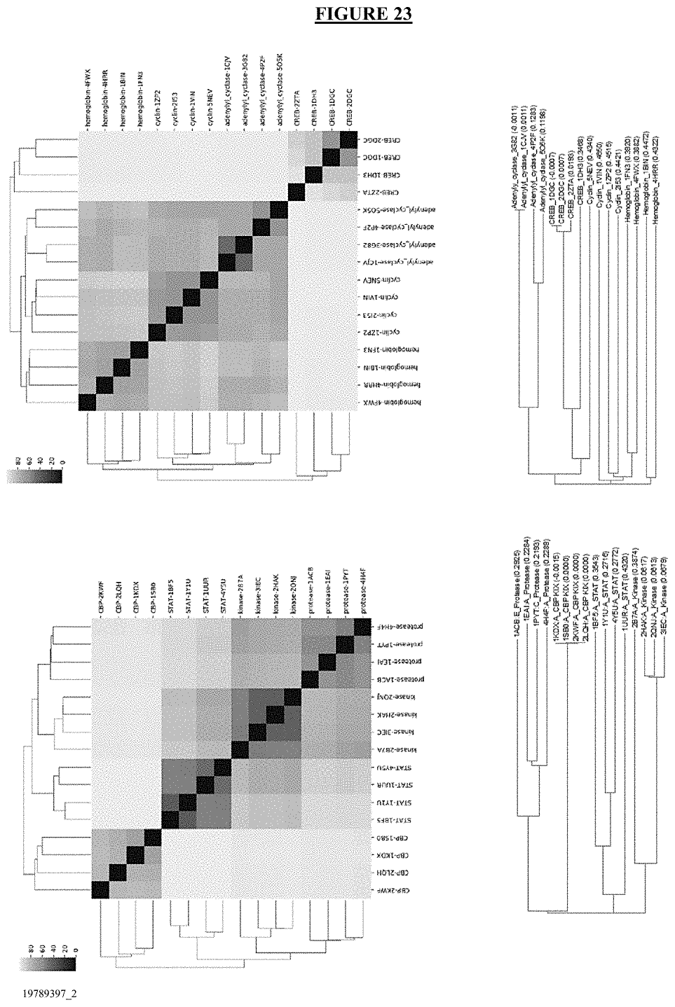

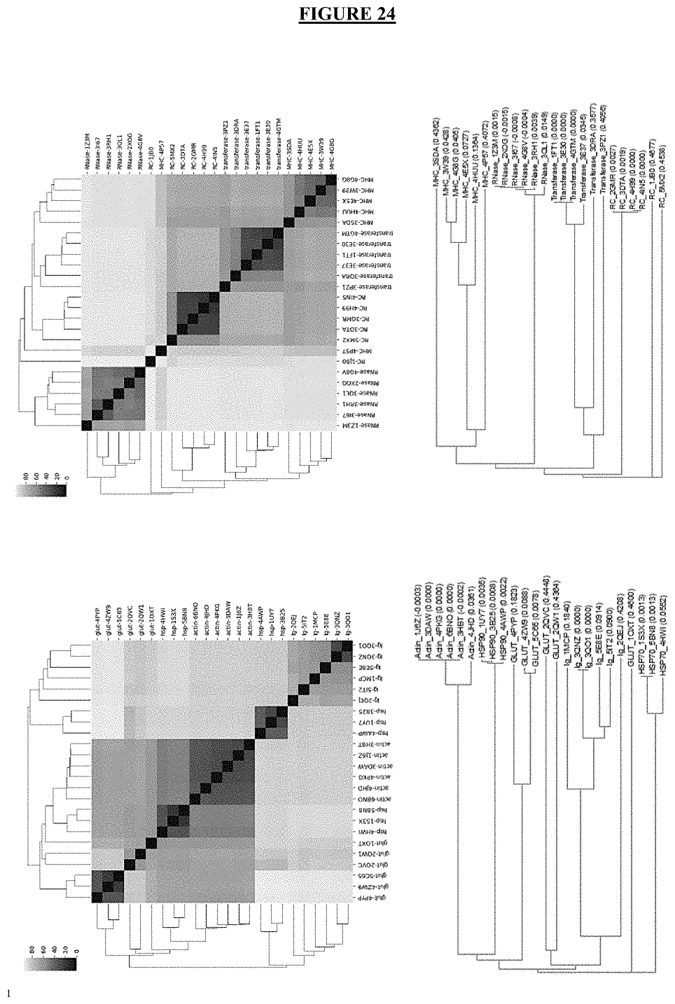

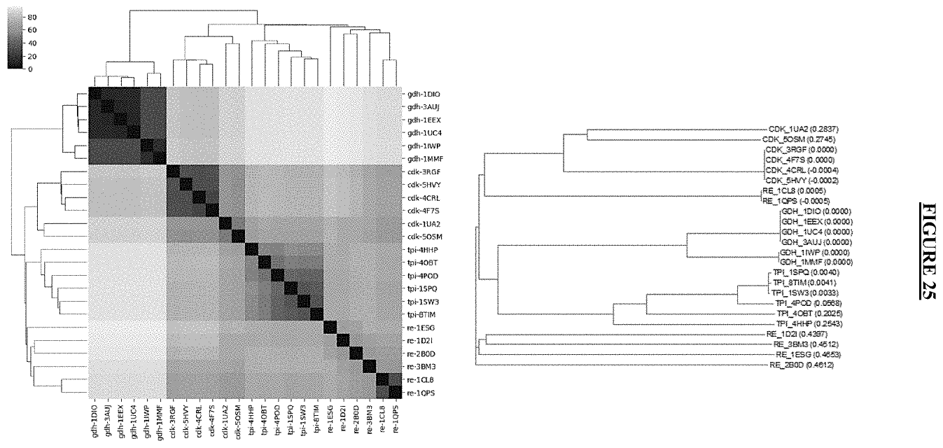

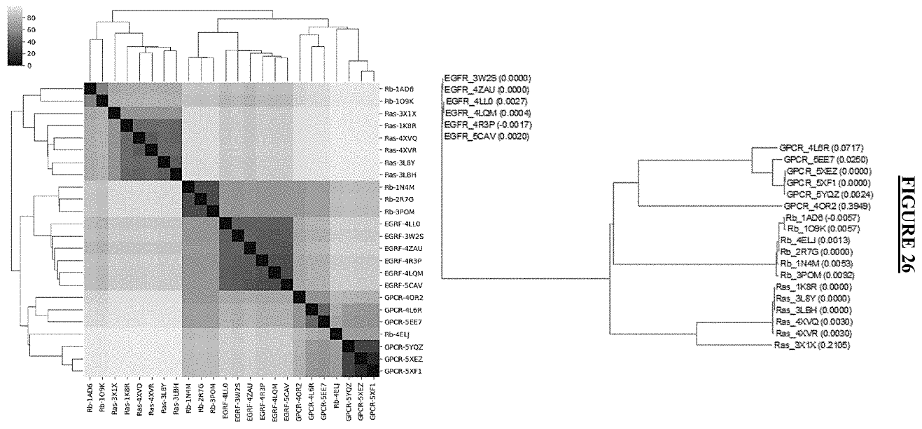

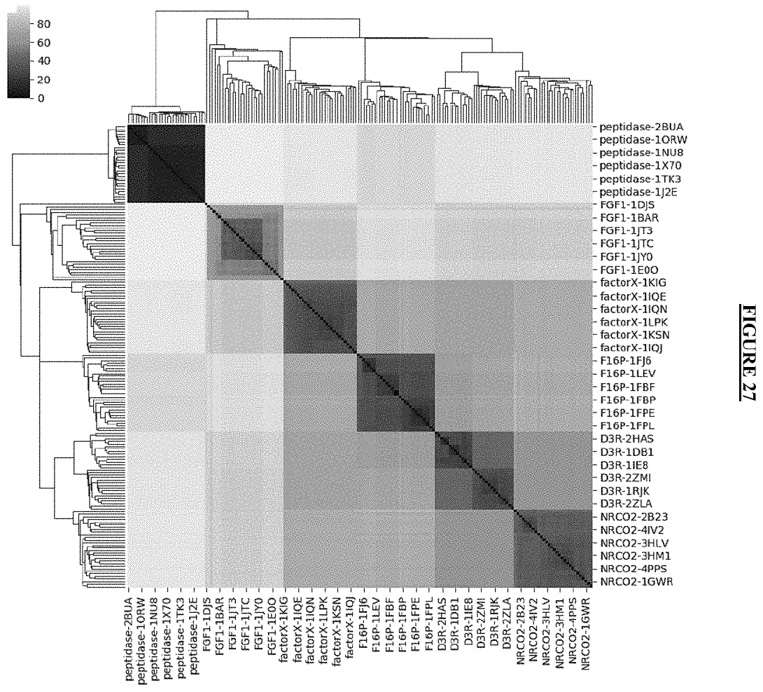

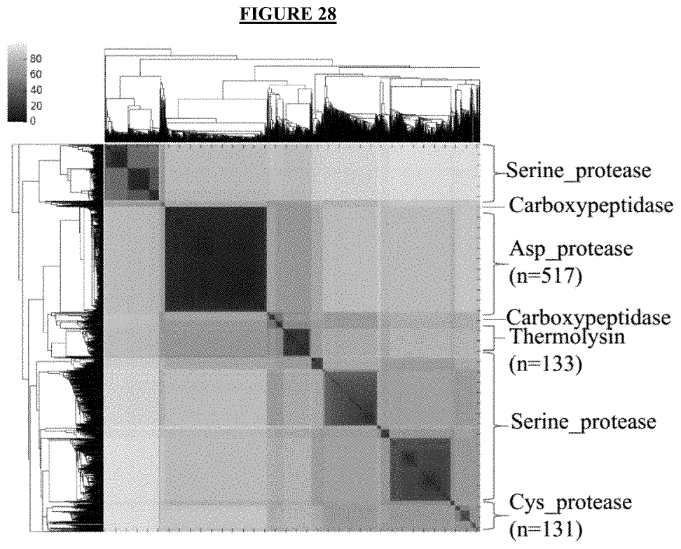

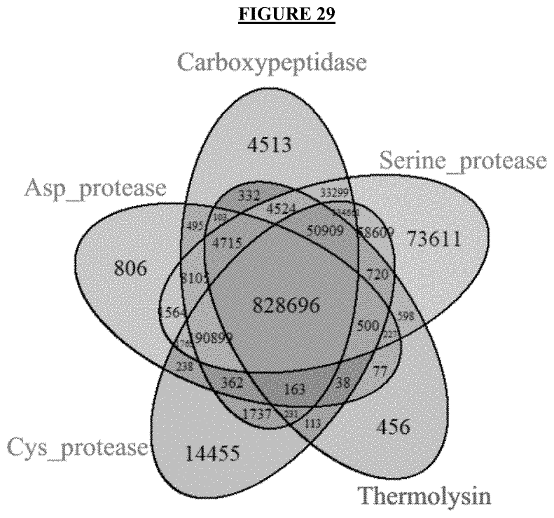

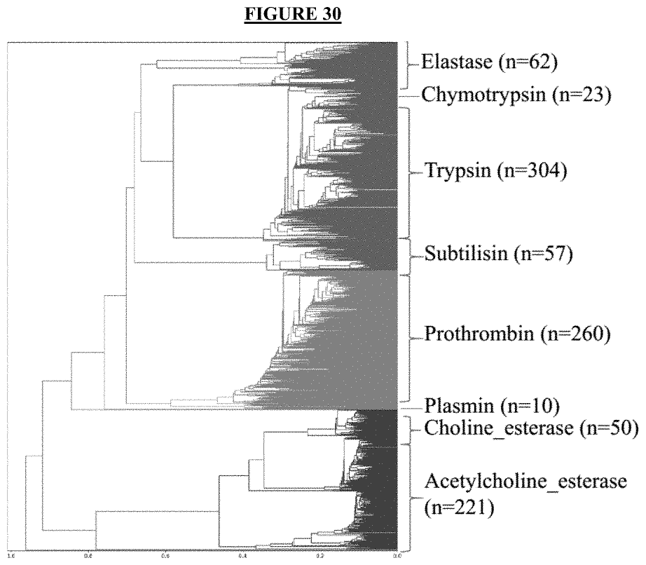

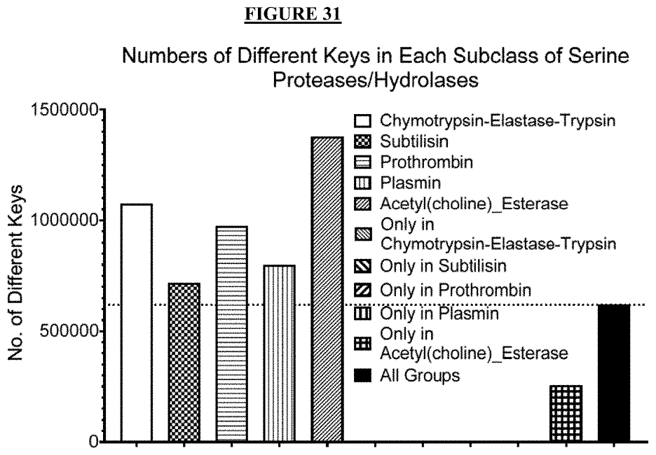

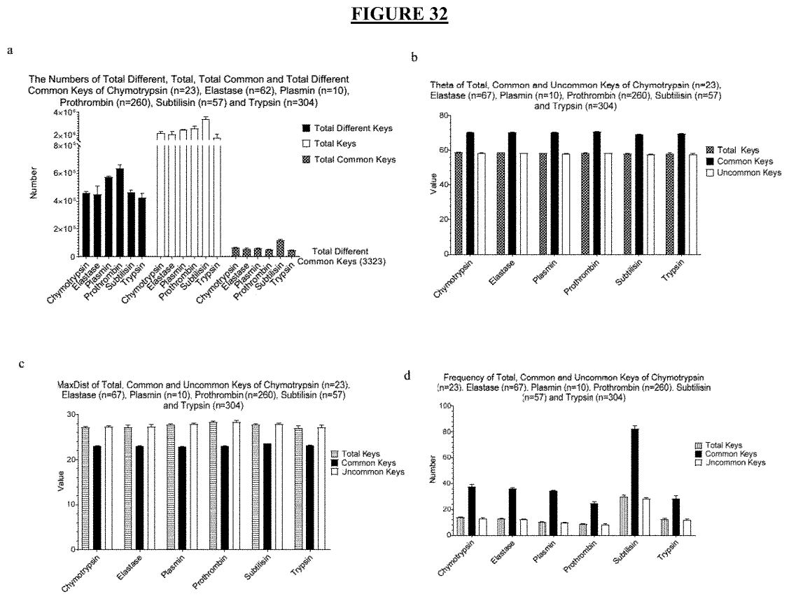

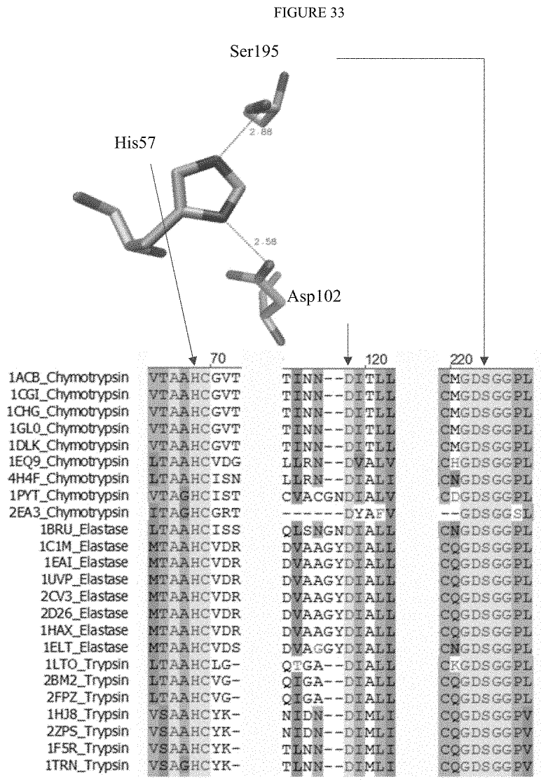

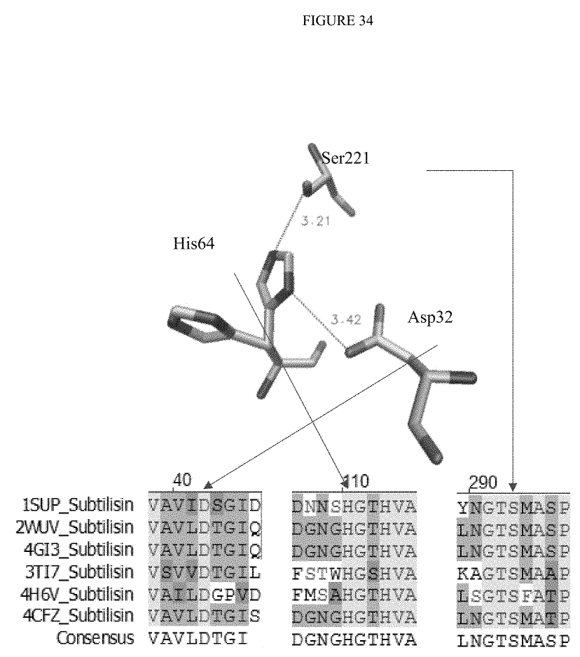

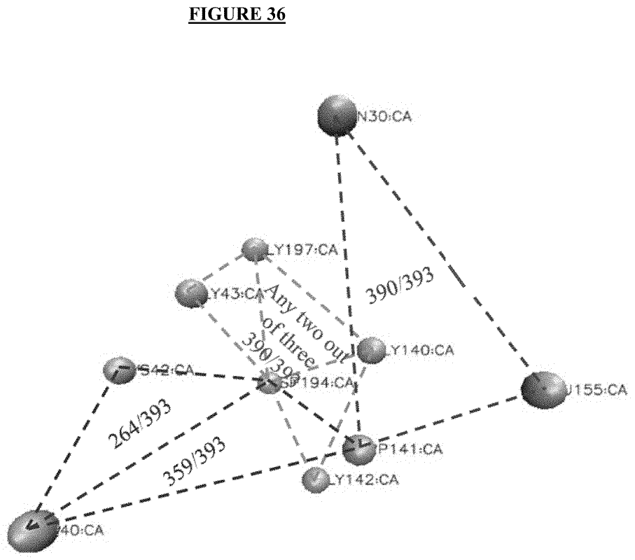

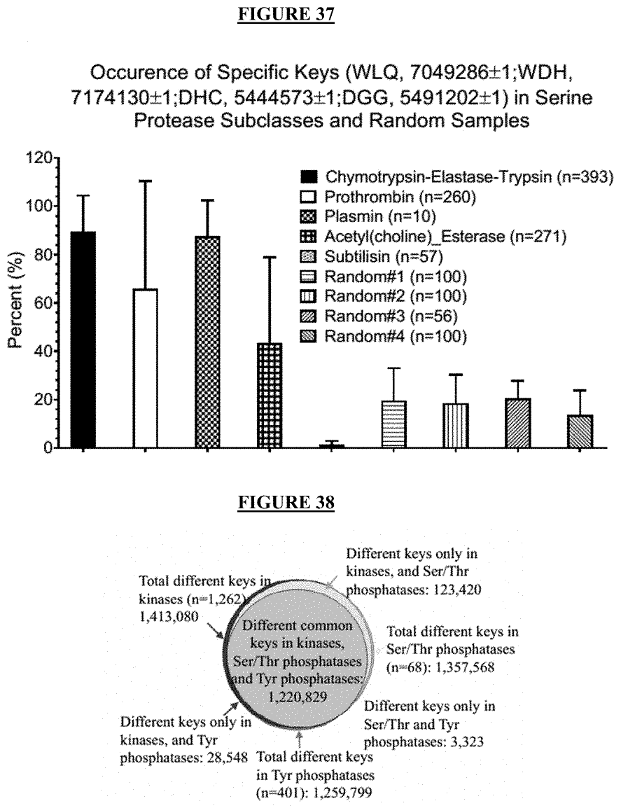

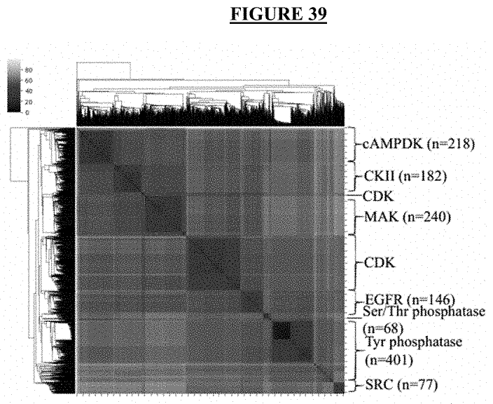

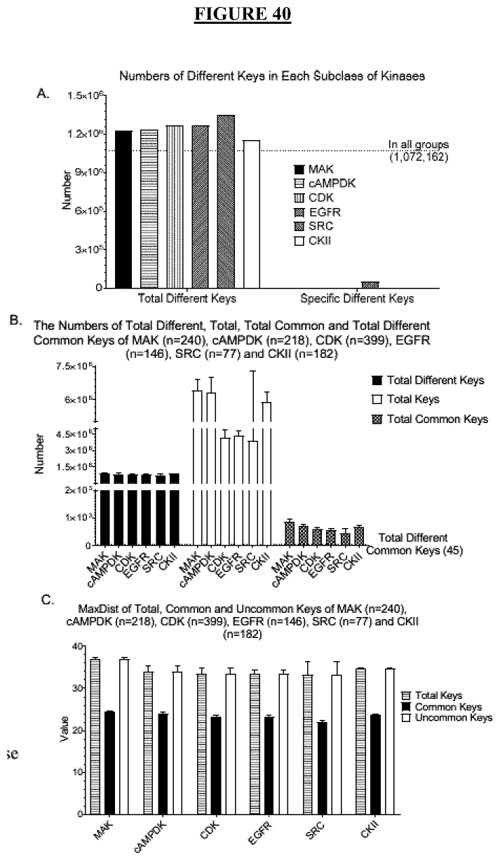

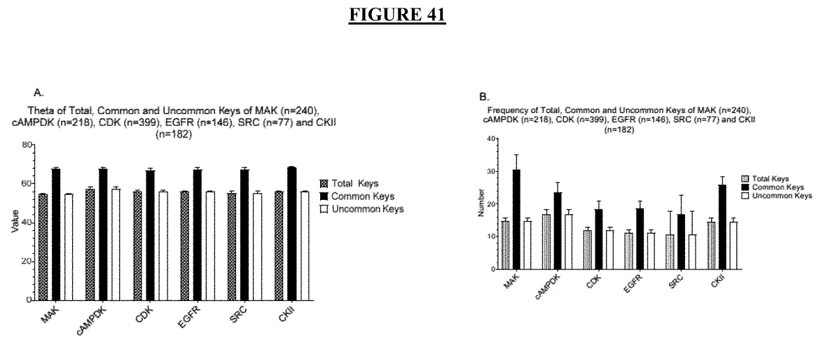

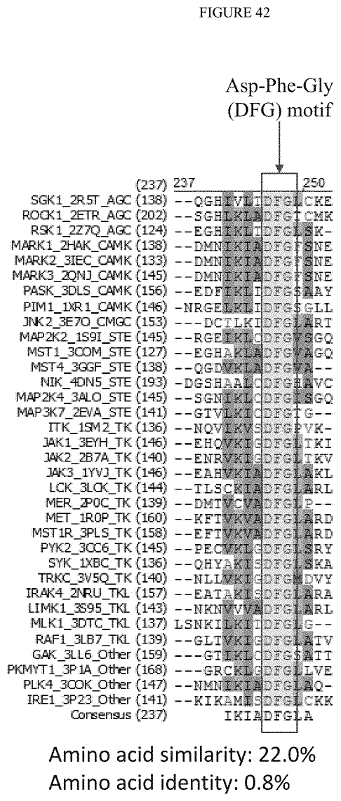

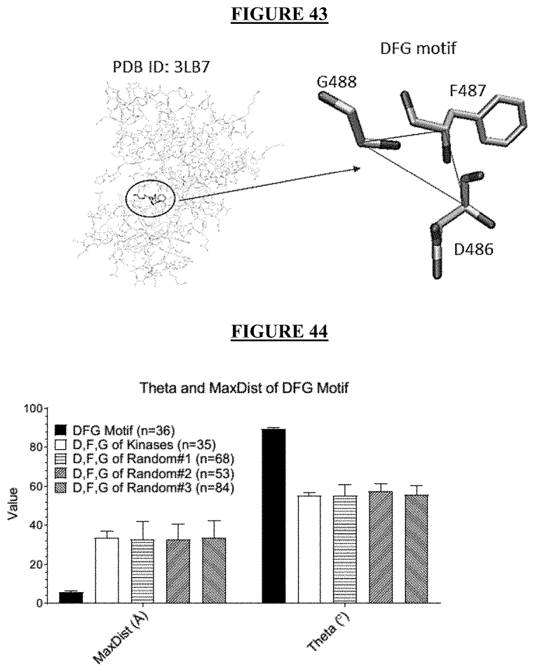

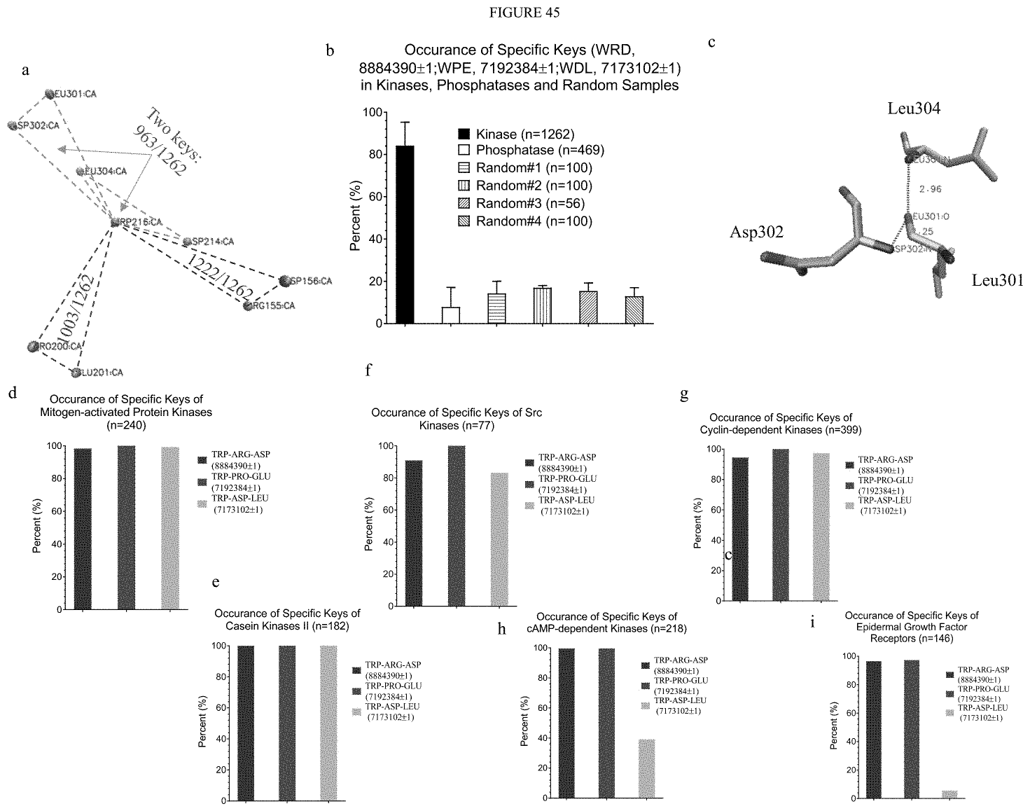

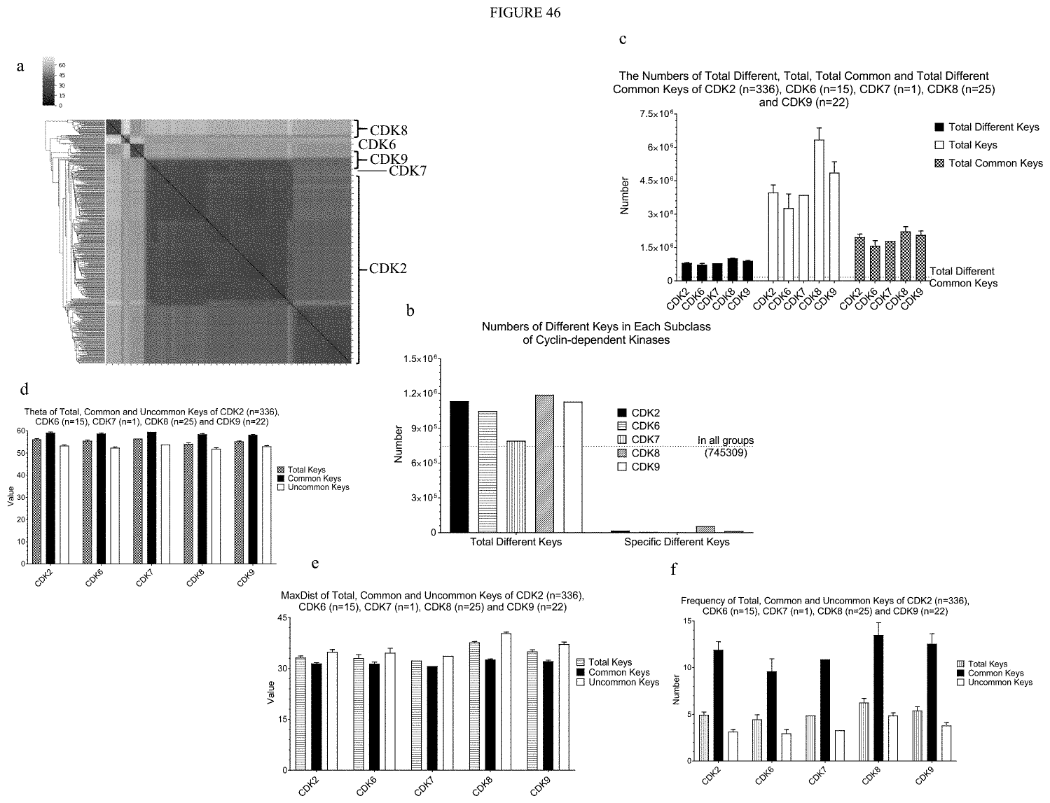

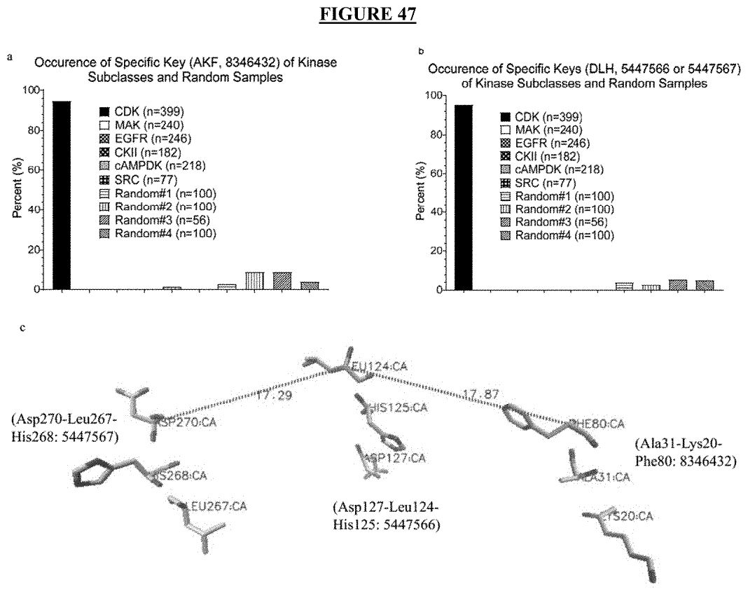

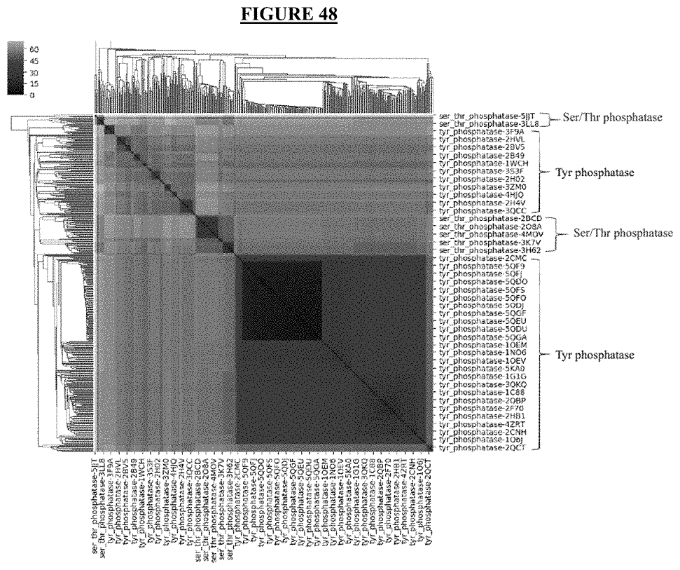

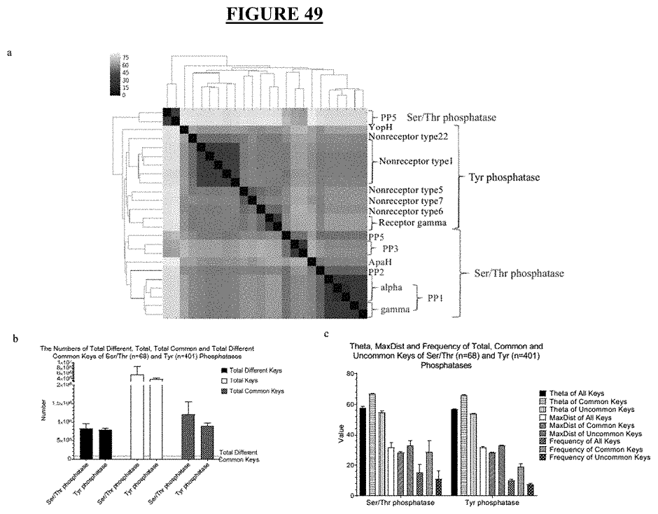

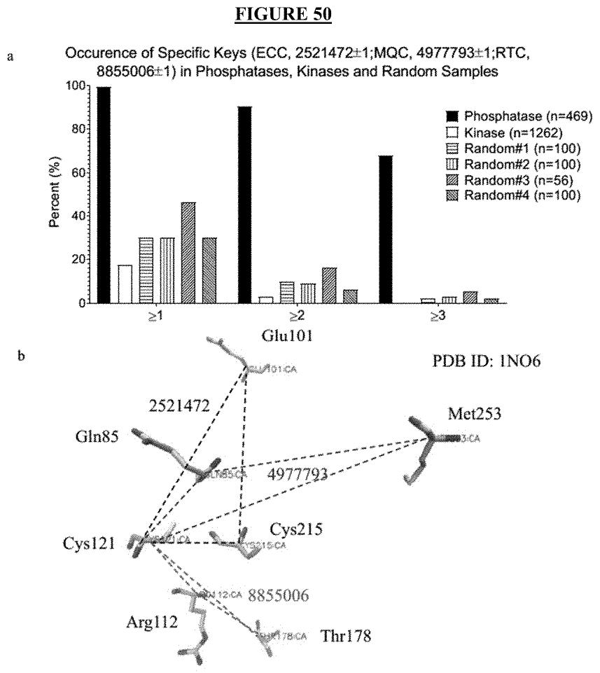

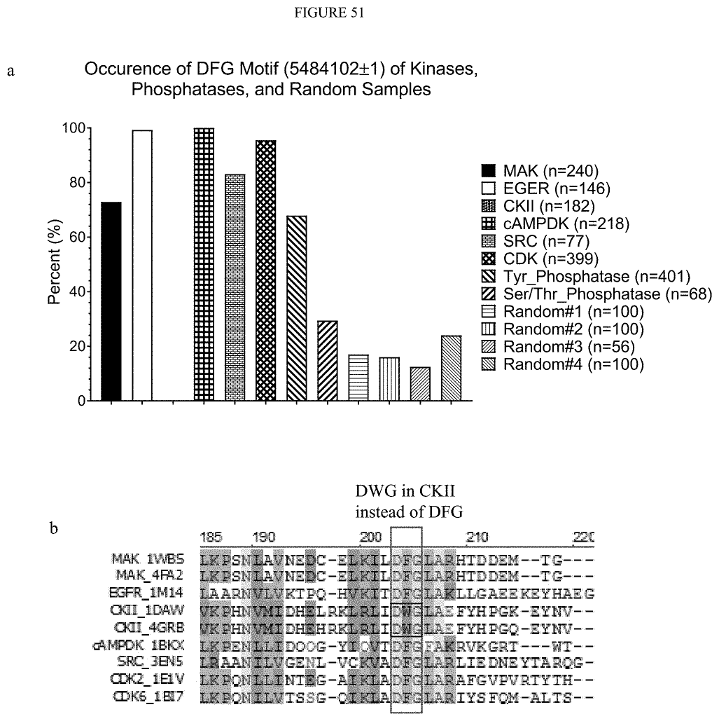

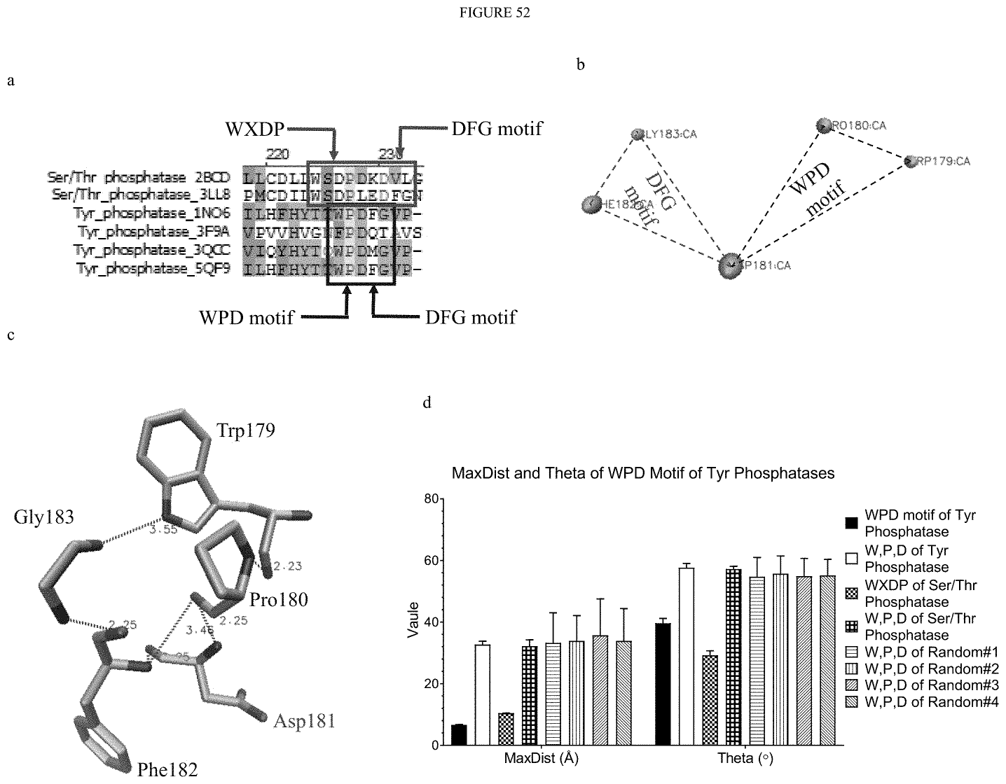

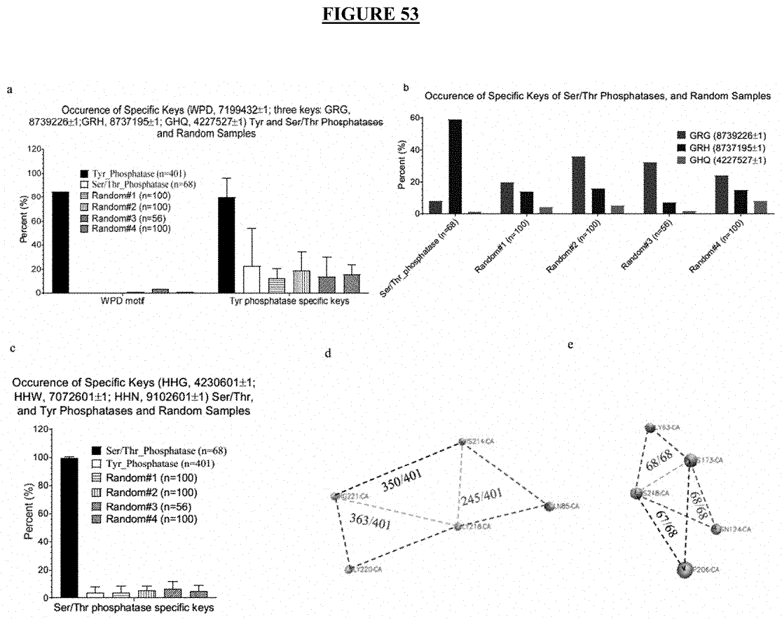

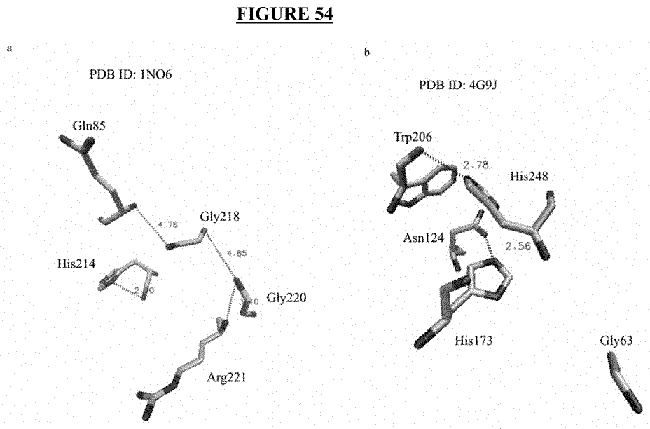

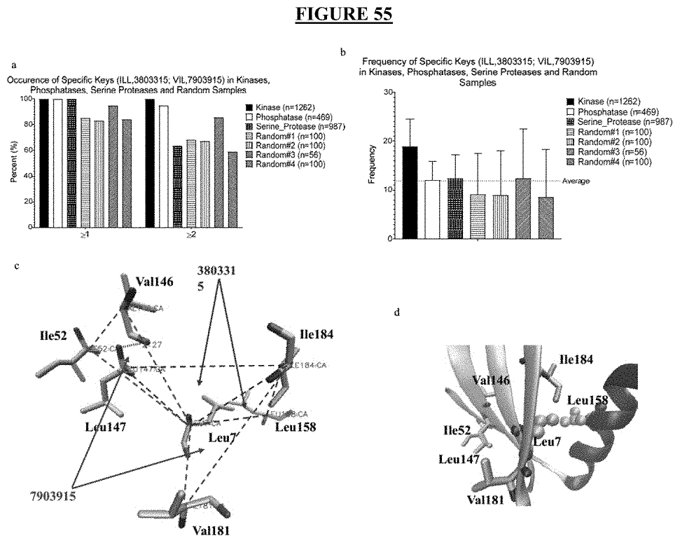

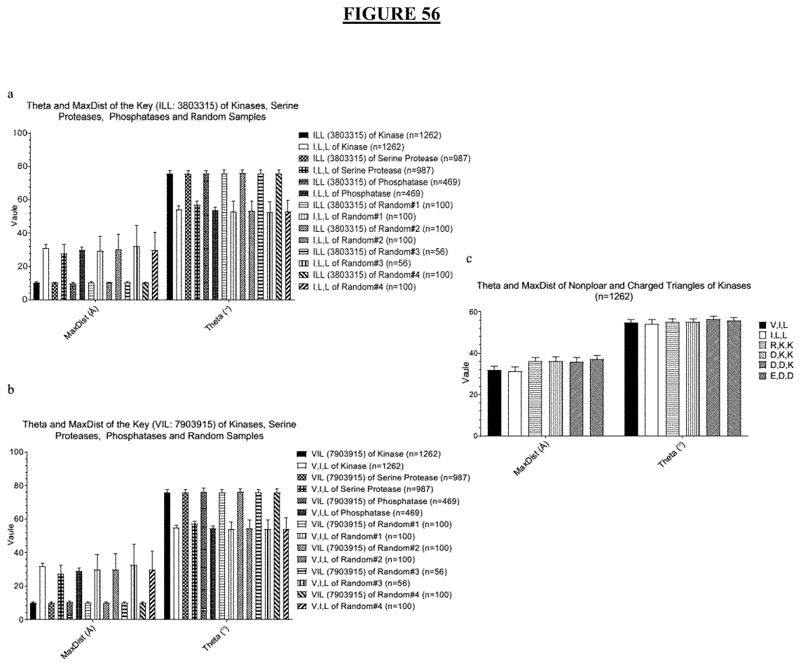

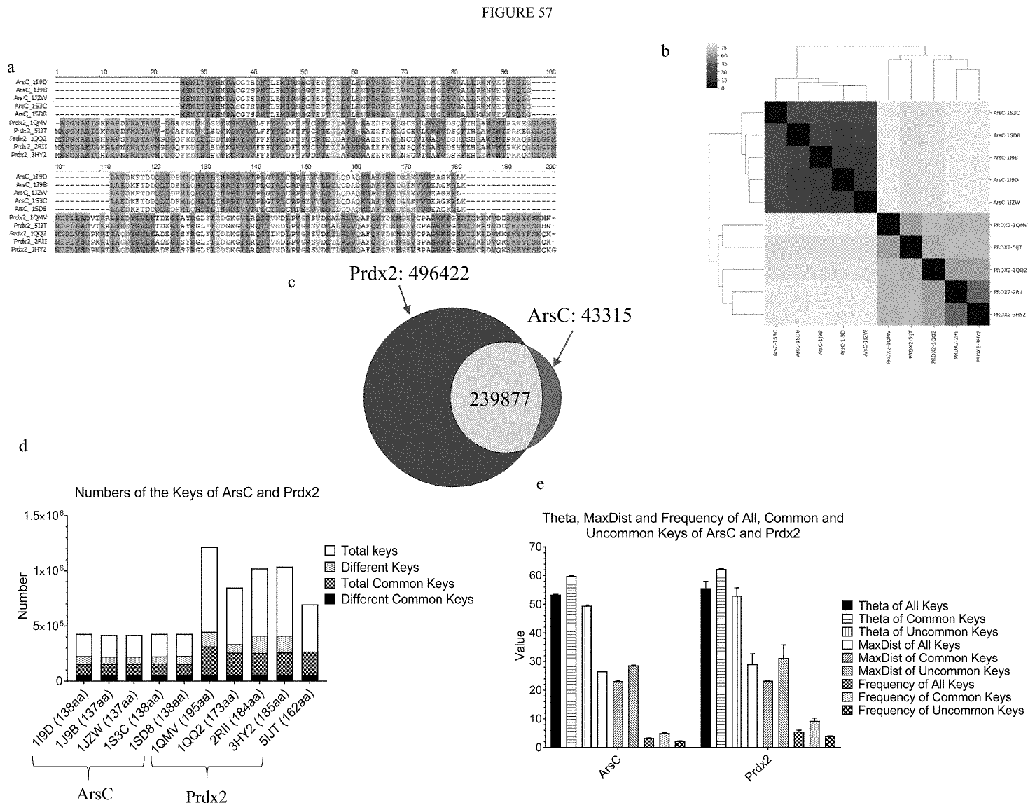

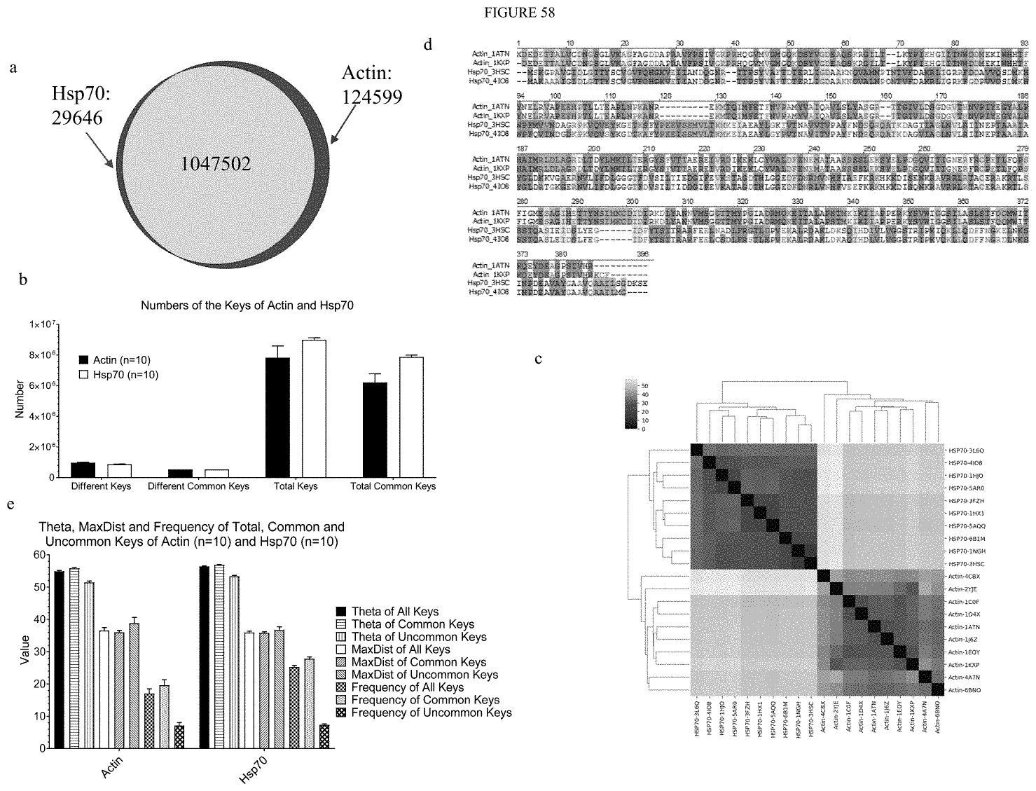

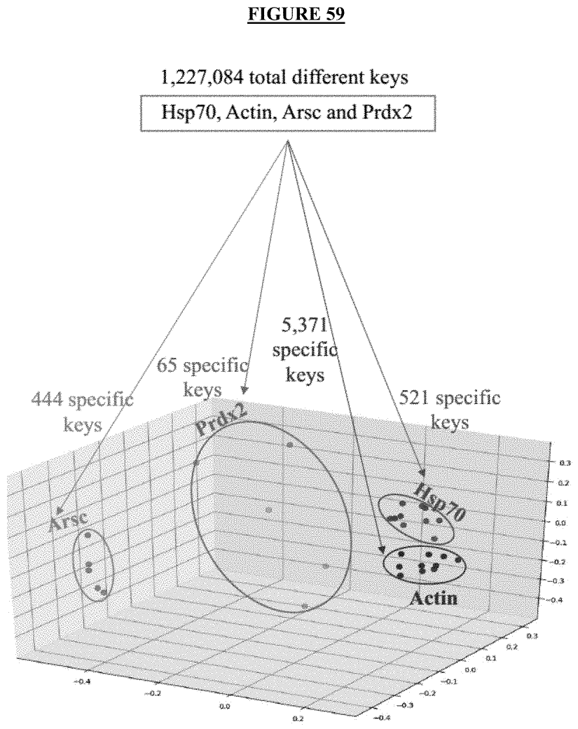

Take for example, the DFG motif (FIG. 4) which is found in protein kinase. DFG is an evolutionarily conserved triple of amino acids that can be seen in sequence alignment. The conserved triple is seen in sequence alignment FIG. 12.

It may also be desirable to find larger motifs or to focus on subgroups within the protein. Protein kinases can be grouped into various classes based on several criterion as shown previously. Longest sub-structure from locally conserved sub-structures for a given class of kinase could give insights into various motifs that may be longer than three amino acids. Algorithm 4 is used to find the structural motifs from longest sub-structure.

In Table 10, Table 11, Table 12, Table 13, and Table 14, and Table 15 the various structural motifs found in kinase classes, AGC, STE, TKL, CAMK, CMGC and TK, respectively are shown. These motifs may be non-contiguous in the sequence. So, these Tables give the examples of proteins and the position of the motifs in the sequence.

### Example Conclusions

The comparison of key distribution between randomly selected keys and locally selected keys revealed that the differences lie in the distribution of length. The locally selected keys are concentrated between 0 and 10 angstrom implying that the functional groups are more closely placed in the space. The cluster analysis between sequence and structure emphasizes that the structural classification is closer to the ideal classification compared to the sequence-based classification.

### Multi-Class Hierarchical Classification

The instant method can also be used in some embodiments for hierarchical protein classification; each level in the hierarchy can have several labels and may have some structural variation. Proteins have a natural structural hierarchy, thus any protein structure comparison or alignment algorithm must have the ability to perform protein classification. The evolutionary, structural, and functional distance between two proteins determines the structural hierarchy. There may be several parts of the proteins that are structurally and functionally independent with respect to the rest of the protein. Such functionally-independent sections of a protein are called domains. In some applications, the classification of protein domains into their respective hierarchical classes is of greater interest than classifying the entire protein, due to their conserved functionality. TSR 3-D-based structural hashing provides a representation of structural nuances of the proteins. And the TSE 3-D keys can be used as the protein attributes for producing correct hierarchical classification of the domain structures.

Most previously-known classifiers are designed for binary classification tasks and none can directly handle hierarchical classification. Multi-class hierarchical classification has previously been handled as a combination of several flat-binary classifiers. Flat classification is the simplest and most commonly used approach to classify protein structures. It simulates hierarchical classification, but does not retain the hierarchical information.

Performance of TSR 3-D is comparable to several other methods in flat-protein structure classification. However, to overcome the inherent shortcomings of flat classification, a new method, Attribute Selected—Local Classifier per Parent Node (“AS-LCPN”), is described herein. This method performs attribute selection based on decision tree at every node, including the root node. The hierarchical classification outperforms flat classification by at least 1.3% average accuracy.

In this embodiment, TSR 3-D is used to define structural units for each protein domain, as explained previously. Key generation function is used to generate unique keys for each structural unit. The entire protein domain is then represented by a set of triples of key-value pairs. The key captures some structural characteristic and the value is the number of times that key occurs in a given protein. It is desirable to use these representative keys for each domain to effectively perform structural classification of protein domains. “Class” as a variable in the hierarchical classification is referred to a group of structurally or functionally related proteins not necessarily of common evolutionary origin.

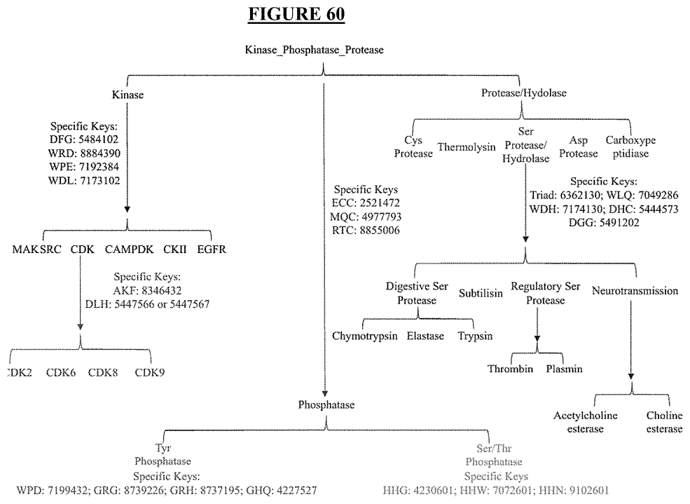

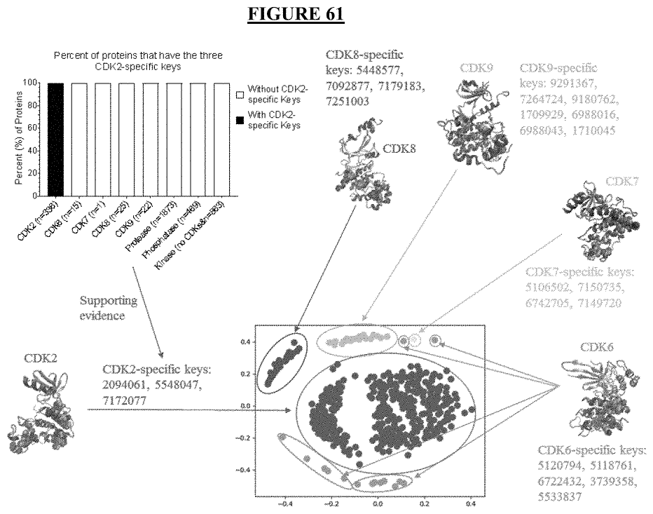

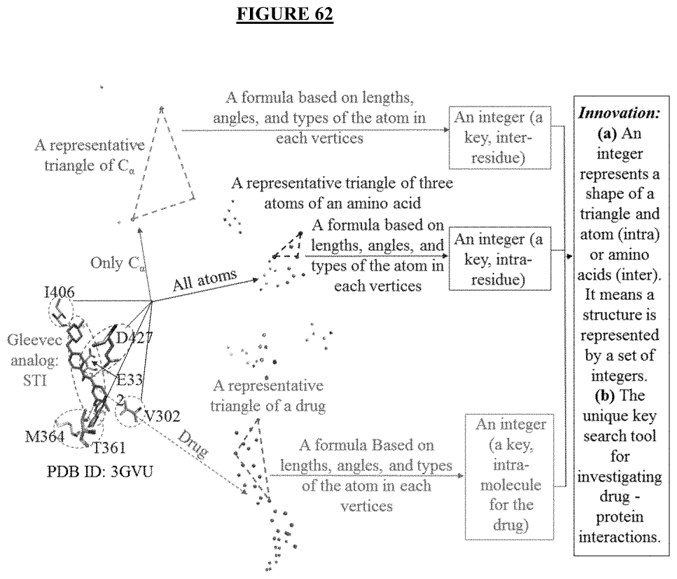

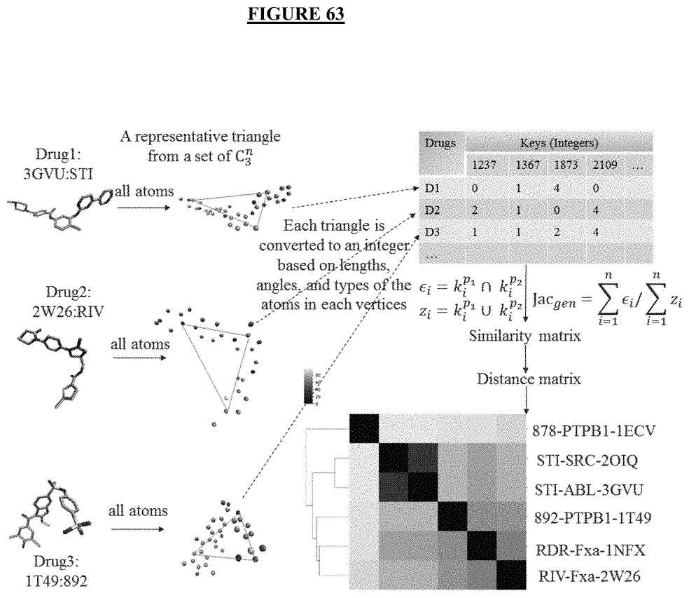

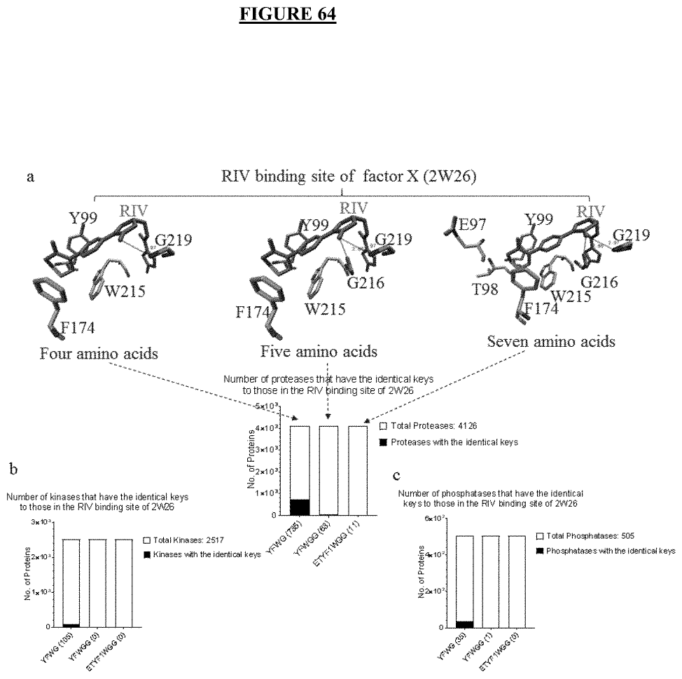

For classification the cross-validated k-nearest neighbor algorithm as known in the art and as illustrated in FIG. 13 is used. Turning to the FIG. 13, TOP 1, TOP 2, TOP 3 represent the search. Objects with (T) marking are test instances. For black star, the instance with correct class is in TOP 1 of the search. For five-point grey star, the instance with correct class is in TOP 2 of the search. For grey triangle the instance with correct class is in TOP 3 of the search.

Let c(test) be the class of test instance, c(train)(1), c(train)(2), c(train)(3) be the classes of training instance ranked 1, 2, and 3 respectively. A test instance is considered correctly classified if For k=1, c(test) is found in c(train)(1); For k=2, c(test) is found in c(train)(1) or c(train)(2); For k=3, c(test) is found in c(train)(1) or c(train)(2) or c(train)(3).

For the purpose of understanding the Method and System for Comparing Proteins in Three Dimensions, references are made in the text to exemplary embodiments of a Method and System for Comparing Proteins in Three Dimensions, only some of which are described herein. It should be understood that no limitations on the scope of the invention are intended by describing these exemplary embodiments. One of ordinary skill in the art will readily appreciate that alternate but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

Reference throughout this specification to features, advantages, or similar language does not imply that all of the features and advantages that may be realized should be or are in any single embodiment. Rather, language referring to the features and advantages is understood to mean that a specific feature, advantage, or characteristic described in connection with an embodiment is included in at least one embodiment. Thus, discussion of the features and advantages, and similar language, throughout this specification may, but do not necessarily, refer to the same embodiment.

Furthermore, the described features, advantages, and characteristics may be combined in any suitable manner in one or more embodiments. One skilled in the relevant art will recognize that the method or system may be practiced without one or more of the specific features or advantages of a particular embodiment. In other instances, additional features and advantages may be recognized in certain embodiments that may not be present in all embodiments.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment.

It should be understood that the drawings are not necessarily to scale; instead, emphasis has been placed upon illustrating the principles of the invention. In addition, in the embodiments depicted herein, like reference numerals in the various drawings refer to identical or near identical structural elements.

### Example 2

This invention also provides novel methods for converting keys into knowledge. In one embodiment, the invention provides an alternative way to calculate keys by grouping amino acids with similar structure and chemical properties together.

Key Generation

For every protein, C_{α} atoms from its PDB file were selected. All three lengths and angles of all possible triangles formed by C_{α} were calculated. Each C_{α} of the 20 amino acids was assigned a unique integer identifier in the range (4, 5, . . . , 23). The integer is transformed IDs to lᵢ₁, lᵢ₂ and lᵢ₃ for vertices of triangle i based on the rule-based label-determination. This transformation ensures that two corresponding triangles receive the same integer IDs. Once lᵢ₁, lᵢ₂ and lᵢ₃ are determined for triangle i, The method calculates θ₁ using the equation No. 1 and θ_{A} based on θ₁ values. (FIG. 14).

$${θ}_{1} = {cos}^{- 1} ( ( {d}_{13}^{2} - {( \frac{{d}_{12}}{2} )}^{2} - {d}_{3}^{2} ) / ( 2 \times ( \frac{{d}_{12}}{2} ) \times {d}_{3} ) ) \\ {θ}_{Δ} = \{ \begin{matrix}{θ}_{1} & if θ \leq 90 ° \\ 180 ° - {θ}_{1} & otherwise\end{matrix} \qquad Equation No . 11$$

Where

d₁₃: distance between lᵢ₁ and lᵢ₃ for triangle i

d₁₂: distance between lᵢ₁ and lᵢ₂ for triangle i

d₃: distance between midpoint of lᵢ₁ and lᵢ₂, and lᵢ₃ for triangle i

Once labels: lᵢ₁, lᵢ₂ and lᵢ₃ and θ_{Δ} are determined, The method uses the Equation No. 12 to calculate key for each triangle.

k=θ_{T}d_{T}(lᵢ₁−1)m²+θ_{T}d_{T}(lᵢ₂−1)m+θ_{T}d_{T}(lᵢ₃−1)+θ_{T}(d−1)+(θ−1) Equation No. 12

where
m: the total number of distinct labels
θ: the bin value for the class in which θ_{Δ} falls to achieve discretization using adaptive unsupervised iterative discretization
θ_{T}: the total bin number of distinct discretization level for angle representative
d: the bin value for the class in which D falls to achieve discretization using Adaptive Unsupervised Iterative Discretization where D is used to achieve scale sensitivity and is given by the distance between lᵢ₁ and lᵢ₂
d_{T}: the total bin number of distinct discretization level for length representative

The determination of bin values and bin numbers will be discussed in the section of Results. The method refers the value of θ_{Δ} as Theta and D as MaxDist. In summary, the key value assigned to a triangle is a function of lᵢ₁, lᵢ₂, lᵢ₃, Theta and MaxDist. In the context of protein structures, the use of MaxDist is a scale factor, since, without MaxDist, two triangles of the same shape, but of different size (similar triangles), could not be distinguished; that is, they will be assigned the same key value.

Protein Structure Similarity and Distance Calculation.

The Generalized Jaccard coefficient measure is applied, Equation No. 13, for the calculation of similarity between two proteins.

Jac_{gen}=Σᵢ₌₁ⁿϵᵢ/Σᵢ₌₁ⁿzᵢ Equation No. 13
where n is the total number of unique keys in proteins p₁ and p₂
Equivalence c for a given key kᵢ in two different proteins p₁ and p₂ is defined as ϵᵢ=kᵤ^{p}^{₁}∩kᵢ^{p}^{₂} where ∩ is defined by the minimum count of the same keys.
Difference z for a given key kᵢ in a pair of proteins is defined as zᵢ=kᵢ^{p}^{₁}∪kᵢ^{p}^{₂} where ∪ is defined by the maximum count of the same keys. The count of a key is the number of times that key occurs (occurrence frequency) within a protein.

A variant of the Generalized Jaccard coefficient measure may also be used, which is referred to herein as the modified Generalized Jaccard coefficient measure, Equation No. 14, to calculate similarity.

mJac_{gen}=Σᵢ₌₁ⁿϵᵢ/min(Σᵢ₌₁ⁿzᵢ, max(Nₚ₁,Nₚ₂)) Equation No. 14

Where Nₚ₁ is a total number of key in p₁

Nₚ₂ is a total number of key in p₂

Once a similarity matrix is generated, the distance matrix is generated simply by each value in similarity matrix subtracted by 1. The protein structure clustering is based on Average Linkage Clustering. The complexity of multiple dimensional relations among 3-D structures are reduced and represented by Multidimesional Scaling method. ClustalW module built in Vector NTI is then applied to conduct pairwise sequence alignments. Structural images were prepared using the Visual Molecular Dynamics (VIVID) package.

Determine Bin Numbers of Theta and MaxDist for Calculating Keys.

To compare two 3-D protein structures, most current methods convert the (x,y,z) coordinates of amino acids to distances between them, make use of topology, or geometry to represent coordinate information. This embodiment provides a completely different approach, where the coordinate information is ultimately converted to a vector of integers, each corresponding to a triangle that acts as a structural primitive. Hence, this approach for protein structural comparison is considered TSR-based.

First all C_{α} atoms are selected and all possible triangles formed by C_{α} atoms are found (FIG. 15). Second, keys are calculated using Equation No. 12 and key occurrence frequencies. Third, The method quantifies similarity or dissimilarity of two structures using the Generalized Jaccard similarity through computing identical and nonidentical keys, and their frequencies (FIG. 15) or the modified Generalized Jaccard similarity methods. This approach enables u structure-based protein classification, and motif identification and discovery (FIG. 15a). The approach does not require prior superimposition of 3-D protein structures and is customized to be sensitive to size of triangles.

To calculate meaningful keys, the foundation is to design an experiment to determine bin numbers of Theta and MaxDist. To do so, 12 different non-overlapping sample sets from PDB are selected, and each contains 30-50 proteins. For each sample set, all angles and lengths are calculated. Theta-count plots show that count generally increases with the increase in Theta (FIG. 16). The trend is the same, if either all three angles against count or MaxDist against count were plotted (FIG. 17). Both the plots show skewed distribution. Based on the plots of Theta-count and MaxDist-count, sample variations were observed.

Equal width binning method will end up with a different number of triangles, having specified interval of values for Theta or MaxDist, falling in each bin. To maximize the possibility of the same or similar number of triangles in each bin and to ensure that all occurrences of the same value are placed in the same bin, a novel Adaptive Unsupervised Iterative Discretization method was used to calculate the bin boundaries. Within bin variances of Theta and MaxDist for each sample set were calculated for different choices of total number of bins (i.e. bin numbers) (FIGS. 18 and 19). Top five bin numbers with the smallest variances were chosen for each sample set. Two with the greatest bin numbers were selected from top five bin numbers (FIG. 20), and then minimum, medium and maximum bin numbers and bin numbers with the highest frequencies were calculated (FIG. 20). The top three binning results for MaxDist were identified as having bin numbers: 12, 26 and 35 (FIG. 20), and top four binning results for Theta as having bin numbers: 7, 15, 21 and 29 (FIG. 20).

The method was also analyzed to determine independency on rotation and translation. One protein was selected from PDB (PDB ID: 2HAK, Chain A), rotated it 35° and/or translated it 5 Å, and the original structure along with all these transformations yielded identical keys (FIG. 21). This analysis indicates the method should consider identical structures no matter how a structure is rotated or translated. Next, the effect of Theta and MaxDist bins on key frequencies was tested. The number of keys with high occurrence frequencies decreases with increase in Theta or MaxDist bin number (FIG. 22).

To further determine optimum values of bins for key generation, six small protein sample sets and each set contains 16 to 24 proteins in four different protein families with 4 to 6 members per family were identified. All combinations of four Theta bins and three MaxDist bins to determine the bin numbers were used. The data show 29 for Theta bin and 35 for MaxDist bin produced the best result in most cases for clustering these six protein sample sets (FIGS. 23-26). It was found that 21 for Theta bin and 12 for MaxDist bin can sometimes correctly cluster small size proteins (<200 aa) (data not shown). To make sure that Theta 29 and MaxDist 35 are the optimum bin numbers, the method was examined to determine where clustering a large sample set correctly was possible, and the result shows that the clustering of a total 157 proteins, from six families of about 30 proteins in each family, perfectly matches their functional classifications (FIG. 27). Theta 29 and MaxDist 35 were used for all analyses in the following sections.

Proteases, kinases, and phosphatases play essential roles in signal transduction. Mutations of these enzymes are often associated with diseases, and they offer valuable targets in many therapeutic settings. In addition, catalytic mechanism of serine proteases has been well-established. Therefore, the method was employed in the study of proteases and kinases/phosphatases aimed for structure-based protein classification, and motif identification and discovery

### Example 3

Proteases hydrolyze peptide bonds of proteins, and were classified into four major classes: serine, cysteine, aspartate, and metal proteases before 1970 and now extended to six distinct classes. Glutamate and threonine proteases are the two new classes. Nearly all available structures of serine (987), aspartate (517), cysteine (131), and metal (105 carboxypeptidase and 133 thermolysin) proteases from PDB. This data set contains a total 1,873 structures. The result shows a perfect clustering for aspartate, and cysteine proteases and thermolysin. Serine proteases were clustered into two subgroups, and carboxypeptidases were also clustered into two subgroups (FIG. 28). To find out common keys belonging to all protease classes, specific keys for each class, and two or more classes, The method generated a Venn diagram (FIG. 29). The largest is the common key section, a total 828,696 distinct keys common to all classes, ranging from 59.5% of total distinct keys for serine proteases (828,696/1,393,400) to 92.9% for thermolysin (828,696/892,401). The percentage of the keys specific to each class is small, ranging from 0.051% (456 out of 892,401) for thermolysin to 5.3% (73,611 out of 1,393,400) for serine proteases. This observation indicates that different classes share a large fraction of identical or similar triangles, and only small fraction of triangles is needed to distinguish one class from another.

Serine proteases can be divided into two types based on their functions: digestive system (chymotrypsin, elastase, trypsin, subtilisin), and regulatory system (thrombin, plasmin). neurotransmission (acetylcholine esterase and choline esterase). The method included acetylcholine and choline esterases in the study of serine proteases because of their nearly identical catalytic mechanism to serine proteases. Additionally, both acetylcholine and choline esterases, and serine proteases belong to family of hydrolase. They are 500-600 aa in size and larger than digestive and regulatory serine proteases (200-300 aa). A deeper analysis on serine proteases was performed. The method shows eight clusters of serine proteases that agree with their functional classifications (FIG. 30). The result shows the structures of chymotrypsin, trypsin and elastase are more similar. Serine proteases or hydrolases were separated into two groups in previous protease clustering. One of these two groups includes acetylcholine and choline esterases, and the other group contains digestive and regulatory serine proteases. Not surprisingly, the subclasses of serine proteases share a large fraction of common keys, and the number of the keys specific for each subgroup, except the group of acetylcholine and choline esterases, is small (FIG. 31). The exception for acetylcholine and choline esterases is probably due to their larger protein size. Searching common keys belonging to every protein of serine protease subclasses, except acetylcholine and choline esterases, yields only very small fractions are Common keys regardless of whether key frequency is considered (2.4% out of total keys by average) or not (0.65% out of total different keys by average) (FIG. 32a). Those Common key have greater average Theta (FIG. 32b) and smaller average MaxDist (FIG. 32c) than Uncommon keys. On an average, frequency of those Common keys is two to three times higher than that of the Uncommon keys (FIG. 32d).

In conclusion, the method is able to perform accurate clustering of serine proteases, and different subclasses share high percent (59.5-92.9%) of the common keys. In contrast, only small portion of the keys, called Common keys, are present in every protein, suggesting high structural variations among proteins. The substructures corresponding to the Common keys have distinct features, e.g. Theta, Maxdist, and frequency, from those corresponding to the Uncommon keys.

Next, the method's ability to successfully identify known motifs was demonstrated. The active site, Triad, of serine proteases has been well-studied. It contains three amino acids: His57, Asp102 and Ser195 for human chymotrypsin (PDB ID: 4H4F). Trypsin and elastase have corresponding His, Asp and Ser residues that can be aligned well with chymotrypsin (FIG. 33). However, subtilisin (PDB ID: 1SUP) has an identical Triad (Asp32, His64 and Ser221), but a different order at amino acid sequence level (FIG. 34).

The keys for the Triad of chymotrypsin, trypsin, elastase and subtilisin were calculated, and they all have identical or nearly identical keys, demonstrating the success of the method in the identification of Triad. Next, the question of “What are the unique features of the Triad triangle compared with all other triangles formed from His, Asp and Ser?” was examined. To answer it, Theta and MaxDist for Triad and all possible His-Asp-Ser triangles was calculated. The calculations show that Triad has much shorter MaxDist and larger Theta than the average of all possible His-Asp-Ser triangles of serine proteins, and three protein samples randomly selected from PDB (FIG. 35).

The success of the study on Triad provides a foundation for the next step of new motif discovery. Amino acid sequences of digestive, regulatory and neurotransmission serine proteases are diverse and no amino acids are conserved. At the structural level, four different keys were found, a total five keys: one key of 7049286 (Trp-Leu-Gln), one key of 7174130 (Trp-Asp-His), one key of 5444573 (Asp-His-Cys) and two keys of 5491202 (Asp-Gly-Gly). A representative of these keys of a serine protease (PDB ID: 4H4F) is shown is FIG. 36 High percent of digestive serine proteases have these five keys (FIG. 37). Specifically, they have a high frequency for 7049286 (390 digestive serine proteases out of a total 393), 7174130 (359/393), and 5491202 (390/393), and a relative low frequency for 5444573 (264/393). Plasmins also have fairly high likelihood to have these five keys; ˜60% of prothrombin, and ˜40% of acetylcholine and choline esterases have these five keys. In contrast, most subtilisins do not have them. To demonstrate those keys are specific for digestive serine proteases, The method came up with four sample sets randomly selected from PDB, and found that ˜20% or less of the proteins from the random samples have them.

Next, the method looks at individual keys, majority of prothrombin, plasmin, and acetylcholine and choline esterases have the keys: 7049286 and 5491202 (Supplementary FIG. 14b-e). About 30-60% of the prothrombin, plasmin, and acetylcholine esterases have 7174130, while nearly all choline esterases do not have it. For the key 5444573, ˜80% of plasmin have it, but majority of the prothrombin, and acetylcholine and choline esterases do not have it. Taken together, the method shows that the five keys are specific for digestive serine proteases. Their presence and the percentage of occurrence of the individual keys can distinguish subclasses of serine proteases. Because these five keys have the potential to be used as one of the features specific for serine proteases, the method can be used to understand if structural relations exist among them. Based on the limited structural analysis (PDB ID: 4H4F), the method shows a hydrogen bond between 5444573 and 5491202, and two hydrogen bonds between 7049286 and 5491202, suggesting salt bridges can bring the keys close.

The method found 1,731 structures of kinases (1,262), and Tyr (401) and Ser/Thr (68) phosphatases from PDB. 1,262 kinase structures can be further divided into 240 mitogen-activated kinases (MAK), 77 Src kinases, 399 cyclin-dependent kinases (CDK), 146 epidermal growth factor receptors (EGFR), 182 casein kinase II (CKII) and 218 cAMP-dependent kinases (cAMPDK). The details including PDB IDs, keys and key frequencies can be found in Supplementary Files. Although kinases and phosphatases have low similarity at amino acid sequence level (Supplementary FIG. 15a), they share a large section of common keys at structural level (FIG. 38). The method can cluster kinases and phosphatases into groups that are highly similar to their functional classifications (FIG. 39). Not surprisingly, kinase subclasses share an even larger section of common keys (FIG. 40a). In contrast, only a small fraction were identified as Common keys at individual protein level (FIG. 40b), indicating a large variation of protein structures among proteins. The method observed differences between Common and Uncommon keys of kinases in term of Theta (FIG. 41a), MaxDist (FIG. 40c) and frequency (FIG. 41b). These differences agree with what The method also observed from serine proteases.

Since the method can distinguish structural differences between subclasses of kinases and phosphatases, it provides a base for more detailed studies on motifs. Most kinases have a DFG motif that plays an important role in regulating its kinase activity. The method performed a sequence alignment of 34 kinases selected from 7 subclasses. The alignment shows low similarity, and only three amino acids, DFG, were aligned together (FIG. 42). A representative DFG motif is shown in Supplementary FIG. 43. DFG motif has its uniqueness of Theta and MaxDist (FIG. 44). DFG is well-defined signature for kinases.

Next, the method was used to identify new kinase signatures using the same approach used for the discovery of new serine protease motifs. The method found three different keys, a total four keys: one key of 8884390 (Trp-Arg-Asp), one key of 7192384 (Trp-Pro-Glu), two keys of 7173102 (Trp-Asp-Leu) that are specific for kinases (FIG. 45a). Greater than 80% of kinases have these four keys, while less than 20% of phosphatases, and four random samples have them (FIG. 45b). The percentage of kinases having the four keys from high to low are 8884390 (1,222/1,262), 7192384 (1,003/1,262) and two 7173102 (963/1,262) (FIG. 45a). Two 7173102 keys could interact with each other through a salt bridge (a hydrogen bond) (FIG. 45c). At the subclass level, greater than 80% of MAK, Src, CKII and CDK have all four keys (FIG. 45d-g). Greater than 90% of cAMPDK and EGFR have 8884390 and 7192384, while ˜40% of cAMPDK and greater than 5% of EGFR have two 7173102 keys (FIG. 45h-i).

CDKs have different types, and 399 structures are from CDK2 (352), CDK6 (8), CDK7 (1), CDK8 (25) and CDK9 (13). The method is able to cluster CDKs reasonably well (FIG. 46a). As predicted based on the previous analyses, all CDKs share a large amount of common keys at the subgroup level (FIG. 46b), and a small amount of Common keys at the individual protein level (FIG. 46c). The differences in Theta, MaxDist and key frequency between Common and Uncommon keys of CDKs (FIG. 46d-f) agree with the observations from other kinases. The method was able to identify three keys specific for CDKs: 8346432 (Ala-Lys-Phe), 5447566 (Asp127-Leu124-His125) and 5447567 (Asp270-Leu267-His268). Greater than 90% of CDKs have these keys while nearly absence or low frequency (<10%) was observed in other kinase subclasses and four random samples (FIG. 47a-b). A representative structure of the three keys is shown in Supplementary FIG. 47c. Three are no close interactions between the keys, as they are separated by more than 17 Å.

Phosphatases catalyze the reversible reaction of kinases. The method has shown the results of structure-based kinase and phosphatase clustering. If the method clusters only phosphatases, Ser/Thr phosphatases were divided into two groups and Tyr phosphatases were also separated into two groups (FIG. 48), suggesting different types inside Ser/Thr and Tyr phosphatase classes. Six proteins from each of the four clusters were selected and examined. The data clearly shows Ser/Thr phosphatases have five types: PP1 (alpha and gamma), PP2, PP3, PPS and apaH. Tyr phosphatases can be divided into receptor and nonreceptor types (Types 1, 5, 6, 7 and 22) and bacterial type (YopH) (FIG. 49a). The phosphatase clustering agrees with their functional classification. In contrast to a large fraction of common keys at the subclass level, Ser/Thr and Tyr phosphatases have a small fraction of Common keys at the individual protein level (FIG. 49b). The method has also identified known and new motifs for kinases, and subclasses of kinases.

The specific keys for phosphatases are first identified, and the keys for Tyr phosphatases and Ser/Thr phosphatases are identified. The method identified three keys: 2521472 (Glu-Cys-Cys), 4977793 (Met-Gln-Cys) and 8855006 (Arg-Thr-Cys) specific for phosphatases. Greater than 90% and ˜70% of phosphatases have at least two keys, and all three keys respectively. As the control, less than 5% and 1% of kinases, and less than 20% and 8% of four random samples have at least two keys and all three keys, respectively (FIG. 50a). A representative structure of the three keys is shown in FIG. 50b. No close interactions between three keys are observed.

It was reported that Tyr phosphatases have a WPD motif that contains catalytic aspartate residue. The method also found high percent of phosphatases have a DFG motif (FIG. 51a). Interestingly, CKII proteins exhibit a lack of the DFG motif (FIG. 51a). The sequence analysis demonstrated CKII have a DWG motif, instead of a DFG motif (FIG. 51b). The observation that many phosphatases have a DFG motif motivated us to look into the details of DFG in phosphatases. The method found that certain Tyr phosphatases have overlapped Asp for WPD and DFG motifs although some Tyr phosphatases have DMG instead of DFG (FIG. 52a). A representative structure of the DFG and WPD motifs is shown in Supplementary FIG. 52b. There are two hydrogen bonds inside the DFG motif, as well as two hydrogen bonds inside the WPD motif. Both motifs are close at amino acid level and structure level (3-4 Å) (FIG. 52a, c). The method also found that Ser/Thr phosphatases have a WXDP sequence and certain Ser/Thr phosphatases have a DFG motif (FIG. 52a). Although it is still questionable whether WXDP is a motif, WPD of Tyr phosphatases and WXDP of Ser/Thr phosphatases have their unique Theta and MaxDist values compared with those of the average Theta and MaxDist values of Trp, Pro and Asp of Tyr and Ser/Thr phosphatases as well as those of random samples (FIG. 52d).

The method identified three Tyr phosphatase-specific keys: 8739226 (Gly-Arg-His), 8737195 (Gly-Arg-Gln), and 4227527 (Gly-His-Gln). Greater than 80% Tyr phosphatases have these three keys, a similar percent observed for having a WPD motif. The control groups: Ser/Thr phosphatases and random samples have relatively high percent (15-25%) to have the three keys (FIG. 53a). Further individual key analysis shows that ˜60% of Ser/Thr phosphatases have 8737195, and 20-30% of random samples have 8739226 (FIG. 53d). The method also found three Ser/Thr phosphatase-specific keys: 4230601 (His-His-Gly), 7072601 (His-His-Trp) and 9102601 (His-His-Asn) (FIG. 53c). The representative structures of Tyr phosphatase-specific keys and Ser/Thr phosphatase-specific keys are shown in Supplementary FIG. 24d-e, and no salt bridges were found between the specific keys for either Tyr or Ser/Thr phosphatases (FIG. 54a-b).

Identification of Common Keys for Proteins.

The method was able to identify Common keys from subclasses of serine protease, and subclasses of kinases and phosphatases. This motivated us to search for the common keys for serine proteases, kinases and phosphatases. The method found two such keys: 3803315 (Ile-Leu-Leu) and 7903915 (Val-Ile-Leu). Nearly 100% of serine proteases, kinases, and phosphatases have one of these two keys (FIG. 55a). Greater than 80% of four random samples have one of the two keys (FIG. 55a). Average frequency of these two keys is between 11 and 12 (FIG. 55b). A representative structure of the two 3803315 and two 7903915 formed by 7 amino acids is shown in FIG. 55c. Six out of seven amino acids locate in a β pleated sheet and the remaining one is from an α helix (FIG. 55d). Even without knowing the the function of these two keys in protein folding, the analysis shows that 3803315 and 7903915 have their specific Theta and MaxDist values (FIG. 56a-b). Hydrophobic amino acids are most likely found in the core of globular proteins. One supportive evidence is from the observation of shorter MaxDist found in the triangles from nonpolar amino acids (e.g. Val, Ile, Leu) compared with the triangle having charged amino acids (Arg, Lys, Glu, Asp) (FIG. 56c). Because the core could play more important roles in protein folding than the protein surface, the initial folding process, or some points during the folding of globular proteins could start from interaction between side chains of Val, Ile or Leu through hydrophobic interaction.

Approximately 200 papers have been published on structural comparison/alignment since 1980. Among these algorithms, DALI, SSAP, CE, VAST, PrlSM, SSM LOCK/LOCK 2, ASSAM/SPRITE, IMAAAGINE, RASMOT-3D PRO, and SPASM have been widely used. Kim and his colleagues constructed a map of the “Protein Structure Space” by using the pairwise structural similarity scores and found that Prdx2 (PDB ID: 1QMV, Chain A) and ArsC (PDB ID: 1J9B, Chain A) have similar structures, and both belong to the GO family 0016491 (oxidoreductase). The DALI algorithm will assign them as structurally different proteins (similarity score: 242.3, Z-score: 1.7, RMSD: 3.5 Å). The sequence alignment shows that Prdx2 and ArsC have low amino acid identity and high similarity (FIG. 57a). The method is able to generate a cluster that agrees with their functional classification (FIG. 57b). These two types of oxidoreductases share a large fraction of the common keys at Prdx2-ArsC level (FIG. 57c) and smaller fraction at the individual protein level (FIG. 57d). The Common keys have shorter MaxDist and higher Theta and key frequency values compared to all and the Uncommon keys (FIG. 57e). Holmes and his colleagues found that Actin (PDB ID: 1ATN) and Hsp70 (PDB ID: 3HSC) have similar structures although there is very little sequence identity between the two proteins. The method shows these two classes share a very large section of the common keys at the class level (FIG. 58a) and a small fraction of the Common keys at the individual protein level (FIG. 57b). The cluster obtained matches their classifications by function (FIG. 57c) and sequence. It is not surprising that they have high amino acid similarity in contrast with a low identity (FIG. 57d). The Common keys have higher frequency than the Uncommon keys (FIG. 57e). The method clearly shows four distinct clusters when the method combined Arsc and Prdx2 with Actin and Hsp70. 65, 444, 521, and 5,371 distinct keys can be used to distinguish Prdx2, Arsc, Hsp70 and Actin respectively from the rest of the classes (FIG. 59). “Protein Structure Space” and DALI methods consider α helices similar. In contrast, the method will show that α helices are different even though they have similar topology if they have different amino acid compositions. “Protein Structure Space” and DALI methods are designed to classify proteins based on their topology while the method is based on geometry and function. The specific keys the method identified allow establishment of structure-based hierarchical relations of proteases, kinases and phosphatases (FIG. 60). A small set of specific keys (from three to seven) were identified for CDK2, CDK6, CDK7, CDK8 and CDK9 (FIG. 61). The results clearly demonstrated that three keys (U.S. Pat. Nos. 2,094,061, 5,548,047, 7,172,077) are specific for CDK2 (Supplementary FIG. 32), and five, four, four, and seven keys are specific for CDK6, CDK7, CDK8 and CDK9 respectively. Even for the proteins with low amino acid similarity, the method found that the high percentage of the common keys at the (sub)class levels. It suggests total variety of protein structures is considerably smaller than the variety of protein sequences agreed with the prediction from the literature. It also indicates there are rooms for us to increase the bin numbers of MaxDist and Theta. In contrast to high percentages of the common keys at class level, the method found low percentages of the Common keys at individual protein levels, demonstrating diversity of protein structures. Unique key calculation, comparison and search features of the method allow not only to build hierarchical protein structure relations, but also to interpret global and local protein structure relations through analyzing keys. All similarity matrices including proteases, kinases and phosphatases can be found in the Supplementary Files

Most structure comparison methods consider protein folds as rigid bodies and quantify the structural similarity based on an average of atomic distances calculated using backbone coordinates. However, certain regions of a protein structure can be prone to variations, which arise due to structural flexibility for certain functions. In the approach, similar, but not identical, triangle could have identical keys due to the bin numbers used in the key calculation. The method used key±1 for motif identification or discovery to allow structural flexibility. The method can also adjust bin numbers to meet the criterion to achieve certain desired structural flexibility.

The method makes it possible to systematically classify the structures available in PDB, to perform structure-BLAST search like BLAST search for amino acid sequences, and to study TSR-based protein and drug, and protein and protein interactions (FIG. 62). Motif discovery will help to understand protein structure and function relations, and extend well-known motifs to unknown motifs. The identified motifs of proteases, kinases and phosphatases can provide guidance for designing specific drugs

The method is an effective novel means for protein structural comparison at global and local levels that promises to assign function to novel protein sequences, to perform structural search and to discover structural motifs. The method currently use only C_{α} atoms, a common practice. However, it involves loss of information with respect to geometries of side chains and structural relationships between side chains, and between side chains and main chains. Side chain information may be incorporated into the current method for achieving more accurate protein structure classification and motif discovery.

Development of a new method of TSR-based 3-D structure representation of drugs, and quantification of drug similarity. The method may be used in one or more embodiments for drug key calculations (FIG. 63). A drug datasets and tools for comparing keys of drugs, and for quantifying similar and distinct keys may also be developed through this method, as well as atom grouping schema for drug key generation.

Prediction of drug and protein interactions using protein and drug key search tools. Tools to identify all amino acids that are likely to interact with drugs (FIG. 64), and TSR-based drug and target datasets are also developed through this method. From earlier studies, it is known that RIV binds thrombin (factor X) and prevents formation of blood clots. They showed that four to seven amino acids were found in the RIV-binding site. The inter-residue key set derived from seven amino acids is more specific to thrombin than the keys derived from four or five amino acids.

1. A method for analyzing three dimensional structures comprising the steps of:
a. assigning a unique numerical value to three non-collinear objects within said three dimensional structure, said three non-collinear objects form a triangle comprising three vertices and a centroid of each said vertices,
b. generating all possible triples of said three non-collinear objects wherein each said three non-collinear objects is represented by the three dimensional coordinates of said centroid,
c. arranging said non-collinear objects by rule-based assignment,
d. calculating a representative angle,
e. calculating a representative edge length,
f. generating a quintuple of features consisting of said unique numerical value of said three non-collinear objects, said representative angle, and said representative edge length,
g. discretizing said representative angle and said representative edge length, and
h. generating at least one key based on said quaintuple of features.

2. The method of claim 1 further comprising the step of using MAD criterion to select said at least one key.

3. The method of claim 1 wherein said discretizing step is performed by Adaptive Unsupervised Iterative-Discretization.

4. The method of claim 1 wherein said three dimensional structure is a protein.

5. The method of claim 4 where said three non-collinear objects are selected from the group consisting of amino acids and amino acid atoms.

6. The method of claim 4 further comprising the step of applying pairwise protein 3-D structure comparison using said at least one key to generate a structural similarity map.

7. The method of claim 5 further comprising the step of classifying proteins hierarchically using Attribute Selected-Local Classifier per Parent Node.

8. The method of claim 1 wherein said assigning, generating, arranging calculating a representative angle, calculating a representative edge length, generating a quintuple of features, discretizing, and generating at least one key are repeated a plurality of times to generate a plurality of said at least one keys and wherein each of said at least on keys is compared to determine motifs.

9. A inter-residue TSR-based structural comparison method comprising:
a. identifying the coordinates associated with a first plurality of atoms of amino acids of a first protein;
b. converting said coordinates to a plurality of triangles, each of said triangles comprising vertices, edge lengths, edge angles;
c. combining said plurality of vertices, edge lengths, and edge angles of said plurality of triangles to create a set of keys for the first protein;
d. identifying the coordinates associated with a second plurality of atoms of amino acids of a second protein;
e. converting said coordinates associated with a second plurality of atoms of amino acids of a second protein to a second plurality of triangles, each of said second plurality of triangles comprising vertices, edge lengths, edge angles;
f. combining said plurality of second triangles' vertices, edge lengths, and edge angles to create a set of keys for the second protein; and
g. comparing the keys representing 2 proteins to determine their similarity using, for example, Generalized Jaccard coefficient;
wherein said keys representing a protein in the group of proteins are integers.

10. A method for representing amino acid 3D structures using intra-residue TSRs comprising:
a. assigning an integer label to each atom type of an amino acid of at least two amino acids, said assigning step comprising using theta bin boundaries selection algorithms developed for inter-residue TSR;
b. applying the Generalized Jaccard coefficient measure for the calculation of similarity between said at least two amino acids to create a similarity matrix;
c. generating a distance matrix by subtracting each value in said similarity matrix by one;
d. determining the bin number of Theta and MaxDist relative to intra-amino acid TSRs;
e. using said bin number of Theta and MaxDist to calculate amino acid similarities.
